(19) United States
(12) Patent Application Publication
(10) Pub. No.: US 2025/0342973 A1
(43) Pub. Date: Nov. 6, 2025

(54) SYSTEMS AND METHODS FOR TEXTURE ANALYSIS OF ULTRASOUND IMAGES AND USES THEREOF

(71) Applicant: Washington University, St. Louis, MO (US)

(72) Inventors: Michelle Oyen, St. Louis, MO (US); Anthony Odibo, St. Louis, MO (US); Ulugbek Kamilov, St. Louis, MO (US); Emily Sheehan, St. Louis, MO (US); Adrienne Scott, St. Louis, MO (US); Patrick Yang, St. Louis, MO (US); Yuyang Hu, St. Louis, MO (US)

(73) Assignee: Washington University, St. Louis, MO (US)

(21) Appl. No.: 19/196,716

(22) Filed: May 1, 2025

Related U.S. Application Data
(60) Provisional application No. 63/641,174, filed on May 1, 2024.

(51) Int. Cl. G16H 50/30; A61B 8/00; A61B 8/08

(52) U.S. Cl. CPC G16H 50/30; A61B 8/0866; A61B 8/5223

(57) ABSTRACT

A computer-implemented system for predicting fetal development, maternal health, and any combination thereof is disclosed that includes at least one processor operatively coupled to a non-volatile memory, wherein the at least one processor is configured to receive an ultrasound image of a placenta of a subject; transform the ultrasound image into at least one texture parameter; predicting the fetal development, maternal health, and any combination thereof based on the at least one texture parameter.

### CROSS-REFERENCE TO RELATED APPLICATIONS

This application claims priority from U.S. Provisional Application Ser. No. 63/641,174 filed on May 1, 2024, which is incorporated herein by reference in its entirety.

### STATEMENT REGARDING FEDERALLY SPONSORED RESEARCH OR DEVELOPMENT

This invention was made with government support under EB028092 awarded by the National Institutes of Health. The government has certain rights in the invention.

### MATERIAL INCORPORATED-BY-REFERENCE

Not applicable.

### FIELD OF THE INVENTION

The present disclosure generally relates to systems and methods for medical imaging texture analysis to predict fetal development and/or maternal health.

### BACKGROUND OF THE INVENTION

Fetal Growth Restriction (FGR) represents one of the most significant and common challenges in obstetrics, affecting an estimated 5-10% of all pregnancies worldwide. This condition, marked by a fetal weight below the 10th percentile for its gestational age, not only poses immediate risks to neonatal health but also has profound implications for long-term conditions such as hypertension, cardiovascular diseases, and diabetes mellitus. At its core, FGR can be attributed to impaired placental function, leading to an inadequate supply of nutrients and oxygen to the developing fetus. Despite its prevalence and impact, traditional ultrasound analyses have predominantly focused on fetal rather than placental health, potentially overlooking key indicators of FGR and related conditions.

Building on the foundational work of ultrasound segmentation and Gray-Level Co-occurrence Matrix (GLCM) analysis applied to kidney diagnostics, past research changes the application of these computational techniques to the placenta. The segmentation and GLCM analysis, as demonstrated in prior studies on kidneys, have proven effective in identifying tissue characteristics and variances that are not discernible to the naked eye. For instance, in the study of transplanted kidneys, segmentation techniques were able to isolate specific regions of interest, enabling a detailed examination of texture and speckle distributions. This analysis facilitated the identification of tissue abnormalities with high precision.

### SUMMARY OF THE INVENTION

Among the various aspects of the present disclosure is the provision of computational medical imaging analysis systems and related methods to predict fetal development and/or maternal health.

Briefly, therefore, the present disclosure is directed to computer-implemented systems and methods for predicting fetal development and/or maternal health based on texture analysis of medical images.

In one aspect, a computer-implemented system for predicting fetal development, maternal health, and any combination thereof is disclosed that includes at least one processor operatively coupled to a non-volatile memory, wherein the at least one processor is configured to receive an ultrasound image of a placenta of a subject; transform the ultrasound image into at least one texture parameter; predicting the fetal development, maternal health, and any combination thereof based on the at least one texture parameter of the subject. In some aspects, the at least one ultrasound image of a placenta comprises an ultrasound image segmented to isolate the placenta image. In some aspects, the at least one texture parameter is obtained using a texture analysis method selected from a filter-based method, a spectral method, a structural method, a deep learning method, and any combination thereof. In some aspects, the at least one texture parameter comprises at least one metric of a Gray Level Co-occurrence Matrix (GLCM) selected from contrast, dissimilarity, homogeneity, energy, correlation, and any combination thereof. In some aspects, predicting the fetal development, maternal health, and any combination thereof based on the at least one texture parameter of the subject further comprises predicting a fetal development comprising a fetal growth restriction (FGR) condition or a maternal abnormality comprising a severe pre-eclampsia (PE) condition. In some aspects, the FGR or severe PE condition is predicted based on a comparison of at least one metric of a Gray Level Co-occurrence Matrix (GLCM) obtained from the ultrasound image and from a healthy subject, a reference subject with a known FGR condition, and a reference subject with a known severe PE condition. In some aspects, the FGR condition is predicted when the at least one metric of the Gray Level Co-occurrence Matrix (GLCM) is significantly different from a corresponding metric of the healthy reference subject and the reference subject with the known PE condition, and the severe PE condition is predicted when the at least one metric of a Gray Level Co-occurrence Matrix (GLCM) is significantly different from a corresponding metric of the healthy reference subject and the reference subject with the known FGR condition. In some aspects, the FGR is predicted when the contrast of the ultrasound image is lower than a corresponding contrast of a healthy subject; the dissimilarity of the ultrasound image is lower than a corresponding dissimilarity of a healthy subject; the homogeneity of the ultrasound image is higher than a corresponding homogeneity of a healthy subject; and the energy of the ultrasound image is higher than a corresponding energy of a healthy subject. In some aspects, the ultrasound image is a placental image of the subject.

In another aspect, a computer-implemented system for selecting a treatment for a pregnant subject based on an ultrasound image of a placenta of the subject is disclosed, in which the system includes at least one processor operatively coupled to a non-volatile memory. The at least one processor is configured to receive the ultrasound image of the placenta of the subject; transform the ultrasound image into at least one texture parameter; predict a fetal abnormality comprising a fetal growth restriction (FGR) or a maternal abnormality comprising a severe pre-eclampsia (PE) condition Fetal Growth Restriction based on the at least one texture parameter; and recommending the treatment if the FGR or PE condition is predicted, wherein the treatment comprises an FGR treatment or a PE treatment. In some aspects, the at least one ultrasound image of a placenta comprises an ultrasound image segmented to isolate the placenta image. In some aspects, the at least one texture parameter is obtained using a texture analysis method selected from a filter-based method, a spectral method, a structural method, a deep learning method, and any combination thereof. In some aspects, the at least one texture parameter comprises at least one metric of a Gray Level Co-occurrence Matrix (GLCM) selected from contrast, dissimilarity, homogeneity, energy, correlation, and any combination thereof. In some aspects, the FGR is predicted based on a comparison of at least one metric of a Gray Level Co-occurrence Matrix (GLCM) obtained from the ultrasound image and from a healthy subject. In some aspects, an FGR condition is predicted when the at least one metric of the Gray Level Co-occurrence Matrix (GLCM) is significantly different from a corresponding metric of the healthy reference subject and the reference subject with the known PE condition; and a severe PE condition is predicted when the at least one metric of a Gray Level Co-occurrence Matrix (GLCM) is significantly different from a corresponding metric of the healthy reference subject and the reference subject with the known FGR condition. In some aspects, the FGR condition is predicted when the contrast of the ultrasound image is lower than a corresponding contrast of a healthy subject; the dissimilarity of the ultrasound image is lower than a corresponding dissimilarity of a healthy subject; the homogeneity of the ultrasound image is higher than a corresponding homogeneity of a healthy subject; and the energy of the ultrasound image is higher than a corresponding energy of a healthy subject. In some aspects, the FGR treatment is selected from regular monitoring of fetal growth and well-being, recommending delivery before an expected due date, administering a corticosteroid compound to accelerate fetal lung development, maternal hospitalization for closer observation and management, recommending specialized neonatal care, and any combination thereof; and the pre-eclampsia treatment is selected from regular monitoring of maternal blood pressure and fetal well-being, administering an eclampsia-preventing compound comprising magnesium sulphate, administering an antihypertensive compound to control maternal blood pressure, administering a corticosteroid compound to accelerate fetal lung development, and any combination thereof. In some aspects, the antihypertensive compound is selected from children's aspirin, labetalol, methyldopa, nifedipine, and any combination thereof.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a pair of images showing a side-by-side comparison illustrating the transformation from a raw Ultrasound DICOM image to a segmented MHA image.

FIG. 6 is a graph representing the largest box sizes that can fit within segmented images, plotting the box area against the count of images that fit these criteria.

FIG. 7 is a four-panel display of the same placenta, each highlighting different box sizes delineated within the segmented area.

FIG. 8 is a graph that depicts the cumulative distribution functions of intensity data for constant area but varying dimensions, compared against scenarios with fixed dimensions.

FIG. 9 is a graph of the frequency of normalized pixel intensities within segmented placenta images for a fixed area of 6950 pixels.

FIG. 10 is a graph of the frequency of normalized pixel intensities within segmented placenta images for a fixed area of 30000 pixels.

FIG. 11 is a box plot comparison illustrating differences in GLCM texture features-contrast, dissimilarity, homogeneity, energy, and correlation-between Fetal Growth Restriction (FGR) and control groups.

FIG. 12 is a schematic of the digital biomarker project workflow of the present disclosure, which includes starting with initial clinical data, performing Doppler and B-Mode ultrasound imaging, optical coherence tomography imaging, image segmentation analysis and predictive modeling, and providing subsequent clinical recommendations.

FIG. 13A is a graph of the total number of patients recruited as related to the workflow of the present disclosure.

FIG. 13B is a graph of the EFW>10th centile patients recruited as related to the workflow of the present disclosure.

FIG. 13C is a graph of the EFW<10th centile patients recruited as related to the workflow of the present disclosure.

FIG. 13D is a table summarizing the patient population used in the present disclosure.

FIG. 14 is a table summarizing the patient population used in the present disclosure.

FIG. 15A is a graph of the maternal age at delivery of the patient population used in the present disclosure, separated into control, other, near-miss, and FGR groups.

FIG. 15B is a graph showing the percentage of patients with chronic hypertension separated into control, other, near-miss, and FGR groups.

FIG. 15C is a graph of the maternal BMI of the patient population used in the present disclosure, separated into control, other, near-miss, and FGR groups.

FIG. 15D is a graph showing the percentage of patients with pre-pregnancy diabetes separated into control, other, near-miss, and FGR groups.

FIG. 15E is a graph of the gestation time of first ANC of the patient population used in the present disclosure, separated into control, other, near-miss, and FGR groups.

FIG. 15F is a graph showing the percentage of multiparous and primiparous patients separated into control, other, near-miss, and FGR groups.

FIG. 15G is a graph of the gestation time at birth of the patient population used in the present disclosure, separated into control, other, near-miss, and FGR groups.

FIG. 15H is a graph showing the percentage of smoking patients separated into control, other, near-miss, and FGR groups.

FIG. 16A is a graph of the baby birthweight of the patient population used in the present disclosure, separated into control, other, near-miss, and FGR groups.

FIG. 16B is a graph showing the percentage of babies born via C-section separated into control, other, near-miss, and FGR groups.

FIG. 16C is a graph of the Apgar score of the baby population used in the present disclosure, separated into control, other, near-miss, and FGR groups.

FIG. 16D is a graph showing the percentage of male and female babies separated into control, other, near-miss, and FGR groups.

FIG. 16E is a graph of the venous cord pH of the baby population used in the present disclosure, separated into control, other, near-miss, and FGR groups.

FIG. 16F is a graph showing the percentage of babies that needed resuscitation during birth separated into control, other, near-miss, and FGR groups.

FIG. 16G is a graph of the arterial cord pH of the baby population used in the present disclosure, separated into control, other, near-miss, and FGR groups.

FIG. 16H is a graph showing the percentage of babies that needed NICU separated into control, other, near-miss, and FGR groups.

FIG. 17 is a chart representing the logistics of Doppler ultrasound measurements related to the patient population and pregnancy time.

FIG. 18A is a graph of left uterine artery pulsatility index (PI) vs. birthweight from Doppler ultrasound measurements in women with 28-32 week gestational age (GA).

FIG. 18B is a graph of left uterine artery pulsatility index (PI) vs. birthweight from Doppler ultrasound measurements in women with 34-38 week GA.

FIG. 18C is a graph of spiral artery pulsatility index (PI) vs. birthweight from Doppler ultrasound measurements in women with 28-32 week GA.

FIG. 18D is a graph of spiral artery pulsatility index (PI) vs. birthweight from Doppler ultrasound measurements in women with 34-38 week GA.

FIG. 18E is a graph of intervillous space (IVS) peak systolic flow rate (PSV) vs. birthweight from Doppler ultrasound measurements in women with 28-32 week GA.

FIG. 18F is a graph of intervillous space (IVS) peak systolic flow rate (PSV) vs. birthweight from Doppler ultrasound measurements in women with 34-38 week GA.

FIG. 19A is a set of images representing the texture analysis techniques of the present disclosure, which includes shape-based segmentation, first-order analysis of individual pixels, and second-order analysis of pixel relationships.

FIG. 19B is a table of radiomic classes and features related to the texture analysis techniques of the present disclosure.

FIG. 19C is another table of radiomic classes and features related to the texture analysis techniques of the present disclosure.

FIG. 20 is a schematic of the image processing workflow of the present disclosure.

FIG. 21 is a schematic representing the ultrasound image segmentation update methods of the present disclosure.

FIG. 22A is a set of images showing ultrasound image segmentation.

FIG. 22B is a table related to the image segmentation FIG. 22A.

FIG. 23 is a set of images representing the image and mask preprocessing of the present disclosure, which includes mask erosion, ROI normalization, and shadow removal.

FIG. 24 is a table of texture image analysis results of FGR vs. Control.

FIG. 25A is a graph of frequency vs. placenta area related to the empirical distribution function of pregnant women.

FIG. 25B is a graph of the empirical distribution function vs. placenta area on visit 1 of pregnant women.

FIG. 25C is a graph of the empirical distribution function vs. placenta area on visit 2 of pregnant women.

FIG. 26A is a graph of the empirical distribution function vs. the first-order mean on visit 1 of pregnant women.

FIG. 26B is a graph of the empirical distribution function vs. the first-order mean on visit 2 of pregnant women.

FIG. 26C is a graph of the empirical distribution function vs. the GLCM cluster tendency on visit 1 of pregnant women.

FIG. 26D is a graph of the empirical distribution function vs. the GLCM cluster tendency on visit 2 of pregnant women.

FIG. 26E is a pair of images showing less and more bright ultrasound images.

FIG. 26F is a pair of images showing less and more homogeneous ultrasound images.

FIG. 27 is a graph showing that texture analysis features correlate with birthweight.

FIG. 28 is another graph showing that texture analysis features correlate with birthweight.

FIG. 29A is a graph of the empirical distribution function vs. autocorrelation (GLCM, first visit) for non-FGR and severe FGR groups from texture image analysis results.

FIG. 29B is a graph of the empirical distribution function vs. run entropy (GLCM, second visit) for non-FGR and severe FGR groups from texture image analysis results.

FIG. 29C is a graph of the empirical distribution function vs. run entropy (GLRLM, first visit) for non-FGR and severe PE groups from texture image analysis results.

FIG. 29D is a graph of the empirical distribution function vs. run entropy (GLRLM, first visit) for non-FGR and severe PE groups from texture image analysis results.

FIG. 29E is a graph of the empirical distribution function vs. run entropy (GLRLM, first visit) for non-FGR and stillbirth nearmiss groups from texture image analysis results.

FIG. 29F is a graph of the empirical distribution function vs. run entropy (GLRLM, first visit) for non-FGR and stillbirth nearmiss groups from texture image analysis results.

FIG. 30A is a graph of the empirical distribution function vs. dependence variance (GLDM, second visit) for non-FGR and severe PE groups from texture image analysis results.

FIG. 30B is a graph of the empirical distribution function vs. large dependence variance (GLDM, second visit) for non-FGR and severe PE groups from texture image analysis results.

FIG. 30C is a graph of the empirical distribution function vs. gray level variance (GLRLM, second visit) for non-FGR and severe PE groups from texture image analysis results.

FIG. 30D is a graph of the empirical distribution function vs. long run emphasis (GLRLM, second visit) for non-FGR and severe PE groups from texture image analysis results.

FIG. 30E is a graph of the empirical distribution function vs. long run high grey level emphasis (GLRLM, second visit) for non-FGR and severe PE groups from texture image analysis results.

FIG. 30F is a graph of the empirical distribution function vs. long run low grey level emphasis (GLRLM, second visit) for non-FGR and severe PE groups from texture image analysis results.

FIG. 30G is a graph of the empirical distribution function vs. run entropy (GLRLM, second visit) for non-FGR and severe PE groups from texture image analysis results.

FIG. 30H is a graph of the empirical distribution function vs. run length non-uniformity normalized (GLRLM, second visit) for non-FGR and severe PE groups from texture image analysis results.

FIG. 301 is a graph of the empirical distribution function vs. low grey level zone emphasis (GLRLM, second visit) for non-FGR and severe PE groups from texture image analysis results.

FIG. 31 is a schematic showing the methods to quantify microvascular structure from OCT images of samples from pregnant women.

FIG. 32A is a graph of surface area per volume quantification from OCT images of samples from pregnant women separated into control, other, near-miss, and severe FGR groups.

FIG. 32B is a graph of villi thickness from OCT images of samples from pregnant women separated into control, other, near-miss, and severe FGR groups.

FIG. 32C is a graph of number of pores per volume from OCT images of samples from pregnant women separated into control, other, near-miss, and severe FGR groups.

FIG. 32D is a graph of volume fraction from OCT images of samples from pregnant women separated into control, other, pre-eclampsia (PE), and severe PE groups.

FIG. 32E is a graph of villi thickness from OCT images of samples from pregnant women separated into control, other, pre-eclampsia (PE), and severe PE groups.

FIG. 32F is a graph of number of pores per volume from OCT images of samples from pregnant women separated into control, other, pre-eclampsia (PE), and severe PE groups.

FIG. 33 is a schematic showing that OCT measurements of samples from pregnant women correlate with birthweight.

FIG. 34A is a graph of surface area per volume vs. birthweight from OCT samples from pregnant women.

FIG. 34B is a graph of villi thickness vs. birthweight from OCT samples from pregnant women.

FIG. 35 is a graph showing that independent ultrasound and OCT measurements are correlated.

FIG. 36 is a set of charts showing that independent ultrasound and OCT measurements are correlated.

FIG. 37A is a ROC curve for severe FGR cases.

FIG. 37B is a table of severe FGR features.

FIG. 37C is a ROC curve for stillbirth near-miss cases.

FIG. 37D is a table of stillbirth near-miss features (including severe FGR).

FIG. 38A is a schematic of the classification model of ultrasound images.

FIG. 38B is an equation for the weighted binary cross-entropy loss used to compensate for unbalanced data during development of the classification model.

FIG. 39A is a schematic of the digital biomarker vision and clinical implementation.

FIG. 39B is a schematic showing the balance between clinical perspectives as characterized by the number of lives saved and ease of implementation, and data perspectives including the quantity of data and available training data.

FIG. 40A is a schematic of potential timelines for clinical implementation of the methods of the present disclosure.

FIG. 40B is a graph of the distribution of gestation end days for stillbirth near-miss cases.

FIG. 40C is a graph of the distribution of gestation end days for not stillbirth near-miss cases.

FIG. 40D is a pair of graphs of the absolute value of Pearson's correlation coefficient (texture features to birthweight) from 2 separate visits.

FIG. 41A is a schematic representing still-birth near-miss factors.

FIG. 41B is a group of graphs, schematics, and images of supporting data showing the prediction of pathologies that lead to stillbirth that are associated with changes in the microstructure of the placenta.

FIG. 42A is a schematic showing the optimization of the predictive model with various degrees of clinical accessibility.

FIG. 42B is a pair of schematics showing potential research with larger datasets, which includes stillbirth near-miss placenta structural phenotyping and informed design of a mechanistic predictive model.

FIG. 43 is a pair of color-coded charts showing that independent ultrasound Doppler and ultrasound texture measurements are correlated.

FIG. 44 is a color-coded chart showing results of texture analysis of ultrasound images of the segmented placenta.

FIG. 45A is a graph of GE correlation vs. distance for θ=0° using correlation sensitivity analysis of 60% of whole placenta.

FIG. 45B is a graph of GE correlation vs. distance for θ=45° using correlation sensitivity analysis of 60% of whole placenta.

FIG. 45C is a graph of GE correlation vs. distance for θ=90° using correlation sensitivity analysis of 60% of whole placenta.

FIG. 45D is a graph of GE correlation vs. distance for θ=135° using correlation sensitivity analysis of 60% of whole placenta.

FIG. 45E is a graph of GE energy vs. distance for θ=0° using energy sensitivity analysis of 60% of whole placenta.

FIG. 45F is a graph of GE energy vs. distance for θ=45° using energy sensitivity analysis of 60% of whole placenta.

FIG. 45G is a graph of GE energy vs. distance for θ=90° using energy sensitivity analysis of 60% of whole placenta.

FIG. 45H is a graph of GE correlation vs. distance for θ=135° using correlation sensitivity analysis of 60% of whole placenta.

FIG. 46A is a graph of left uterine artery PI vs. gestational age for severe FGR, stillbirth near-miss, other, and control cases.

FIG. 46B is a graph of spiral artery PI vs. gestational age for severe FGR, stillbirth near-miss, other, and control cases.

FIG. 46C is a graph of intervillous space PI vs. gestational age for severe FGR, stillbirth near-miss, other, and control cases.

FIG. 47A is a ROC curve for severe FGR cases using logistic regression.

FIG. 47B is a table of severe FGR features.

FIG. 47C is a ROC curve for stillbirth near-miss cases using logistic regression.

FIG. 47D is a table of stillbirth near-miss features (including severe FGR).

FIG. 47E is another ROC curve for severe FGR cases using logistic regression.

FIG. 47F is another table of severe FGR features.

FIG. 47G is another ROC curve for stillbirth near-miss cases using logistic regression.

FIG. 47H is another table of stillbirth near-miss features (including severe FGR).

FIG. 48A is a table of definitions of stillbirth or stillbirth near-miss cases.

FIG. 48B is another table of definitions of stillbirth or stillbirth near-miss cases.

FIG. 49A is a histogram of gestation end days for women used in the present disclosure (controls=1).

FIG. 49B is a histogram of gestation end days for women used in the present disclosure (SFGR=1).

### DETAILED DESCRIPTION OF THE INVENTION

The present disclosure is based, at least in part, on the discovery of a computational method targeting placental texture analysis in prenatal ultrasound imagery. By shifting the diagnostic focus to the placenta, this method emphasizes the critical role of placental health in pregnancy outcomes, offering a more refined approach to detecting conditions that may influence fetal development and maternal well-being. The software program assesses the texture patterns of placental ultrasound images, identifying subtle variances invisible to the human eye yet indicative of FGR and other prenatal conditions. This shift towards a more quantifiable and standardized evaluation of placental health can potentially significantly mitigate the risks associated with the misdiagnosis or oversight of critical placental issues, marking a major step forward in prenatal care.

By integrating advanced computational techniques within a Python framework, our program has the potential to enhance the accuracy of FGR detection and opens new avenues for earlier and more precise interventions across a spectrum of prenatal diagnosis. This innovative approach underscores the importance of placental analysis in prenatal care, offering a groundbreaking tool that bridges the gap between traditional diagnostics and the need for more detailed, objective, and reliable assessments of fetal health. As shown herein, a computational analysis of placental ultrasound images for the detection of fetal growth restriction is described.

One aspect of the present disclosure provides for a computational method specifically tailored for the analysis of placental texture in prenatal ultrasound imagery. Unlike traditional ultrasound analyses that primarily focus on the fetus, this approach shifts the attention toward the placenta. By employing advanced texture analysis algorithms, this technology examines the texture of placental ultrasound images, facilitating the detection of prenatal and placental conditions that might impact fetal development and maternal health with advanced precision and objectivity.

In some aspects, the present disclosure includes the utilization of an array of texture analysis techniques, including but not limited to filter-based, spectral, structural, and deep learning methods. Each of these techniques allows for the detection of subtle variances in the images that may signify a wide range of fetal conditions. This shift towards a more quantifiable and standardized assessment of placental health aims to mitigate the risks associated with the misdiagnosis or overlooking of crucial placental issues, ultimately contributing to superior prenatal care standards.

In accordance with more aspects, designed for seamless integration into current diagnostic protocols, this technology emphasizes scalability and adaptability. It can be incorporated into existing diagnostic workflows without necessitating significant retraining of medical staff or any investment in new hardware. Moreover, its software-based framework allows for ongoing updates, ensuring that the technology remains at the forefront of placental ultrasound diagnostic innovations.

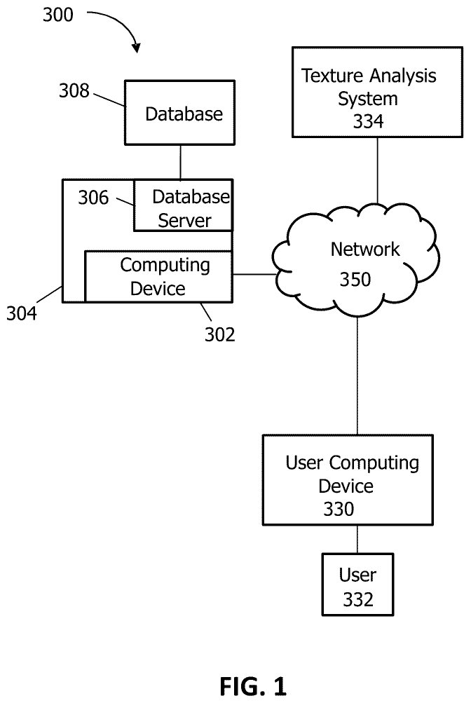

In various aspects, at least a portion of the methods disclosed herein may be implemented using various computing systems and devices as described below. FIG. 1 depicts a simplified block diagram of a computing device for implementing the prenatal screening system and methods described herein. As illustrated in FIG. 1, the computing device 300 may be configured to implement at least a portion of the tasks associated with the disclosed prenatal screening method, including, but not limited to: producing a prenatal screening based on medical imaging data of the placenta of a subject including, but not limited to, ultrasound imaging data, CT imaging data, MRI data, or PET perfusion data. In an exemplary embodiment, the medical imaging data can be an ultrasound image of a placenta of a pregnant woman. The computer system 300 may include a computing device 302. In one aspect, the computing device 302 is part of a server system 304, which also includes a database server 306. The computing device 302 is in communication with database 308 through the database server 306. The computing device 302 is communicably coupled to a user-computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

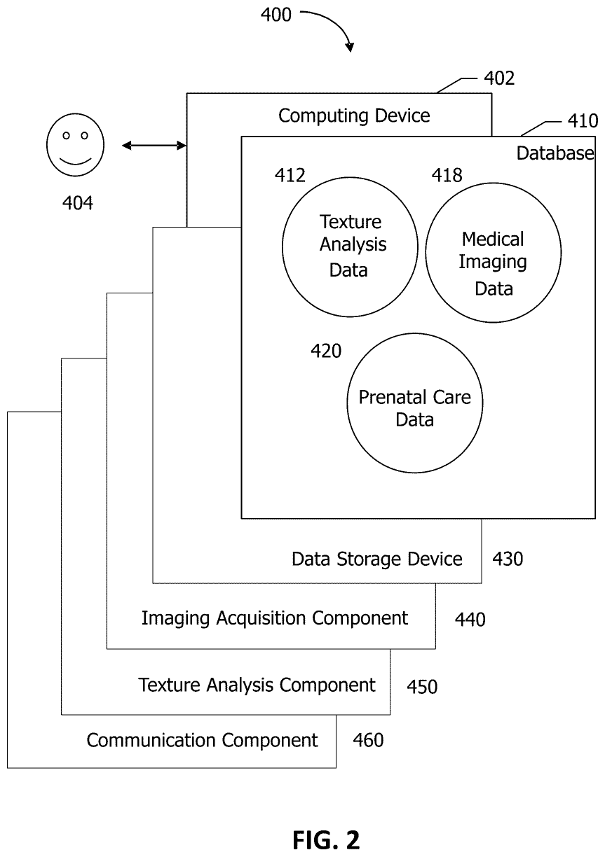

In other aspects, the computing device 302 is configured to perform a plurality of tasks associated with the production of a prenatal screening and method of selecting prenatal care for a woman in need using the prenatal screening system as described herein. FIG. 2 depicts a system 400 with a computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes medical imaging data 418, texture analysis data 412, and prenatal care data 420. Non-limiting examples of medical imaging data 418 include any data characterizing various aspects of placental health including, but not limited to, ultrasound imaging data, CT data, MRI data, and/or PET imaging data. Non-limiting examples of suitable texture analysis data 412 include any values of parameters defining the prenatal screening system, including but not limited to, filter-based, spectral, structural, and deep learning methods to analyze placental imaging data of a pregnant woman as described herein.

Computing device 402 also includes a number of components that perform specific tasks. In the exemplary aspect, computing device 402 includes a data storage device 430, an imaging acquisition component 440, a texture analysis component 450, and communication component 460. Data storage device 430 is configured to store data received or generated by computing device 402, such as any of the data stored in database 410 or any outputs of processes implemented by any component of computing device 402. The imaging acquisition component 440 is configured to produce an analysis of the spectrum of the medical imaging data as disclosed herein. The texture analysis component 450 is configured to detect subtle variances in placental images that may signify a wide range of fetal conditions as described herein.

Communication component 460 is configured to enable communications between computing device 402 and other devices (e.g. user computing device 330 and sequencing system 310, shown in FIG. 1) over a network, such as network 350 (shown in FIG. 1), or a plurality of network connections using predefined network protocols such as TCP/IP (Transmission Control Protocol/Internet Protocol).

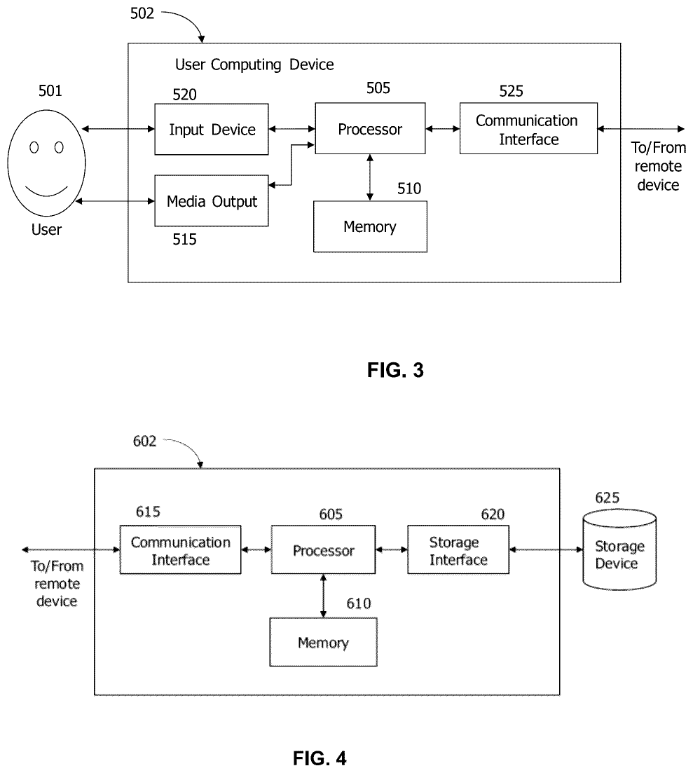

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIGS. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### Therapeutic Methods

Also provided is a process of treating, preventing, or reversing a fetal abnormality, including but not limited to fetal growth restriction (FGR), in a subject in need of an effective medical intervention, so as to maintain a healthy fetus.

Methods described herein are generally performed on a subject in need thereof. A subject in need of the therapeutic methods described herein can be a subject having, diagnosed with, suspected of having, or at risk for developing a fetal abnormality, including but not limited to FGR. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective medical intervention is, for example, an intervention that would cause the desired therapeutic effect in a subject while minimizing undesired side effects. In various embodiments, an effective medical intervention described herein can substantially inhibit a fetal abnormality, slow the progress of a fetal abnormality, or limit the development of a fetal abnormality.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, the medical interventions of the present disclosure can be performed, at a reasonable benefit/risk ratio applicable to any medical treatment, in a sufficient amount to ensure fetal health.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

A medical intervention can occur as a single event or over a time course of treatment. For example, medical intervention can occur daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for FGR and other fetal abnormalities.

A medical intervention can occur simultaneously or sequentially with agents, such as an antibiotic, an anti-inflammatory, or another agent. For example, a medical intervention can occur simultaneously with an agent, such as an antibiotic or an anti-inflammatory. Simultaneous medical intervention can occur through the administration of separate compositions, each containing one or more of a fetal health agent, an antibiotic, an anti-inflammatory, or another agent. Simultaneous administration can occur through the administration of one composition containing two or more of a fetal health agent, an antibiotic, an anti-inflammatory, or another agent. A fetal health agent can be administered sequentially with an antibiotic, an anti-inflammatory, or another agent. For example, a fetal health agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biological or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929;Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Computational Analysis of Placental Ultrasound Images for Detection of Fetal Growth Restriction

In Example 1, a computational method targeting placental texture analysis in prenatal ultrasound imagery is introduced. By shifting the diagnostic focus to the placenta, this method emphasizes the critical role of placental health in pregnancy outcomes, offering a more refined approach to detecting conditions that may influence fetal development and maternal well-being. The software program assesses the texture patterns of placental ultrasound images, identifying subtle variances invisible to the human eye yet indicative of FGR and other prenatal conditions. This shift towards a more quantifiable and standardized evaluation of placental health can potentially significantly mitigate the risks associated with the misdiagnosis or oversight of critical placental issues, marking a major step forward in prenatal care.

By integrating advanced computational techniques within a Python framework, the program has the potential to enhance the accuracy of FGR detection and opens new avenues for earlier and more precise interventions across a spectrum of prenatal diagnosis. This innovative approach underscores the importance of placental analysis in prenatal care, offering a groundbreaking tool that bridges the gap between traditional diagnostics and the need for more detailed, objective, and reliable assessments of fetal health.

### Methods

### Patient Recruitment

Patients with and without Fetal Growth Restriction were recruited for this prospective study, which was approved by the Institutional Review Board at Washington University in St. Louis. FGR was defined as an estimated fetal weight less than the 10th percentile for gestational age, an accepted definition in the United States provided by the Society for Maternal-Fetal Medicine. Patients eligible for the study included women with singleton pregnancies between the ages of 18 and 55 who delivered at Barnes Jewish Hospital in St. Louis. Exclusion criteria included women of ages less than 18 or greater than 55, a presence of a fetal anomaly, twins or higher-order gestations, and patients who did not deliver at Barnes Jewish Hospital. All patient data were de-identified prior to analysis.

### Ultrasound Imaging & Image Segmentation

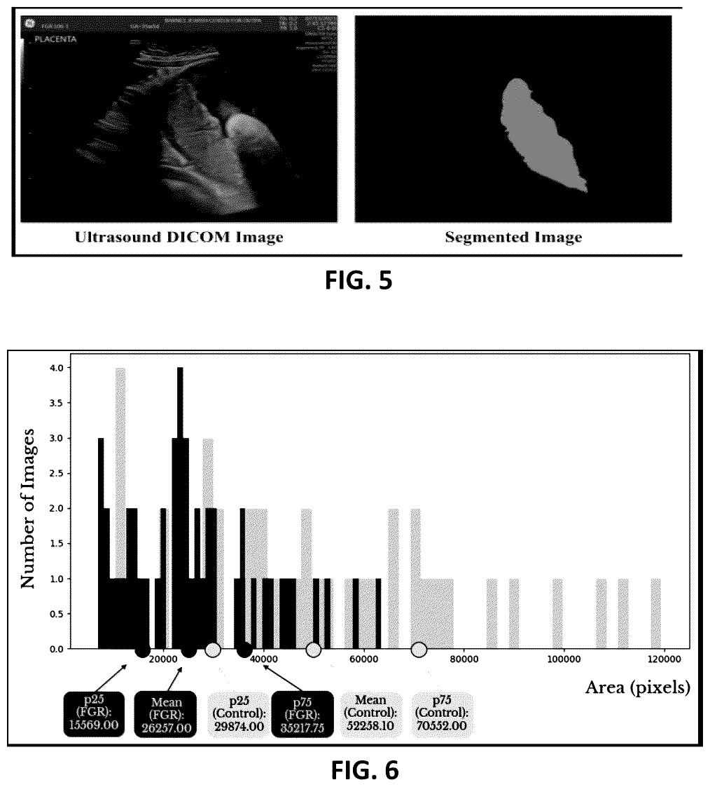

Recruited patients were imaged using Doppler ultrasound between 28-32 weeks of gestation and again at 34-38 weeks of gestation by a trained sonographer. Imaging was completed with a GE HealthCare Volusion E22 machine (GE, Milwaukee, USA). Images of the placenta were acquired by finding the longest axis of the placenta that was visible in one plane in the ultrasound image. Images were saved in the DICOM file format. Images were then manually segmented using ITK-Snap (FIG. 5). Technicians then manually segmented the images using the open-source software, ITK-Snap. In each image, the placenta was identified, traced, and the extracted region was saved as an MHA file, easily processable by Python.

### Software Environment

All analysis was conducted using Python 3.8, with dependencies including NumPy for numerical operations, matplotlib and seaborn for data visualization, and SciPy for statistical analysis. The script design is modular, allowing for easy adaptation and future enhancements.

### Box Size Sensitivity Analysis

The analysis is grounded in the extraction of pixels in rectangular regions of segmented images. The dimensions of the boxes were determined by graphing the area of the largest rectangles inside each image. A Python function, largest_rectangle( ) was written to identify the maximal rectangular area within a binary mask by iterating through the mask to compute consecutive “1”s (true values) in each column, representing heights. For each row, it updates a height array, ‘h’, and then scans each column to find the widest rectangle that can be formed from each starting column, based on these heights. The function calculates the area of each potential rectangle and updates the maximum area found. Finally, it returns the coordinates of the top-left corner and the dimensions (width and height) of the largest rectangle. For visual examination, the rectangles were superimposed on the original DICOM images using the Python Imaging Library (PIL) and saved as JPG files.

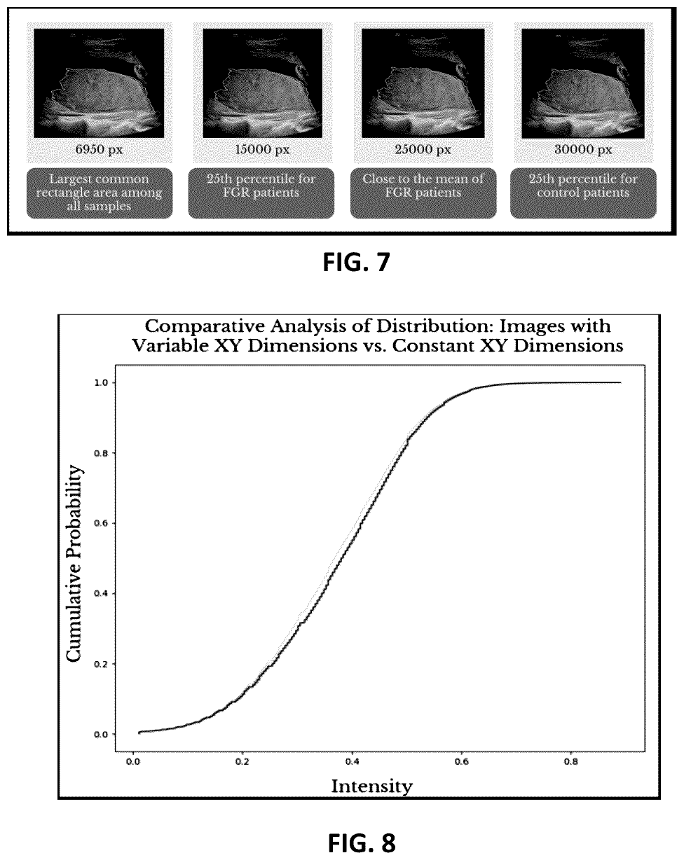

After determining the dimensions of the largest rectangle within each image of the dataset, the sizes were graphed for comparison (FIG. 6). Four areas were chosen for further analysis (FIG. 7). These area sizes include 6,950 pixels, representing the smallest area encompassing the entire dataset; 15,000 pixels, corresponding to the approximate 25th percentile area for fetal growth restriction (FGR) patients; 25,000 pixels, approximating the mean area of FGR patients; and 30,000 pixels, the approximate 25th percentile area for control patients. With each successive iteration focusing on a specific area size, the dataset available for analysis grew smaller as box sizes outgrew the dimensions of segmented images.

For each rectangle size, the ‘extract_fixed_size_rectangle_intensities( )’ function was employed to process the dataset's images. This function identifies and extracts pixel intensities from predetermined rectangular areas within segmented regions, utilizing the DICOM images and their associated binary masks. This methodology was selected following a comparative analysis between fixed-area dimensions and variable dimensions across images with a constant area, which revealed no significant differences in intensity outcomes (FIG. 8). It searches for a rectangle that entirely fits within the segmented region of the binary mask. Upon locating an appropriate rectangle, the function extracts pixel intensities from this area, normalizes these against the maximum intensity of the DICOM image, and returns a flattened array of normalized values along with the rectangle's dimensions.

Once pixel intensities were obtained for patients, they were graphed to compare intensity distributions and spatial characteristics of segmented regions between FGR and control datasets. This comparison utilized histograms, violin plots, and cumulative distribution function (CDF) plots. The intensities extracted from FGR patients were overlaid for comparison.

Additionally, the Gray Level Co-occurrence Matrix (GLCM) technique was leveraged for further analysis. This statistical method, which assesses the spatial relationship of pixels, quantifies the texture of specific image regions. Five metrics to describe GLCM were calculated: contrast, dissimilarity, homogeneity, energy, and correlation. Contrast measures the intensity contrast between a pixel and its neighbor over the whole image, dissimilarity computes the variation in gray levels among neighboring pixels, homogeneity describes the closeness of the distribution of elements in the GLCM to the GLCM diagonal, energy measures the uniformity of the texture, while correlation measures the joint probability occurrence of the specified pixel pairs. The ‘compute_glcm_features( )’ function was written and used, which first standardized the input image to an 8-bit unsigned integer format before calculating the GLCM at a pixel distance of one and an angle of zero degrees, ensuring the matrix was symmetric and normalized. From this matrix, key texture features, including contrast, dissimilarity, homogeneity, energy, and correlation, were extracted, which are essential for the texture analysis.

### Results

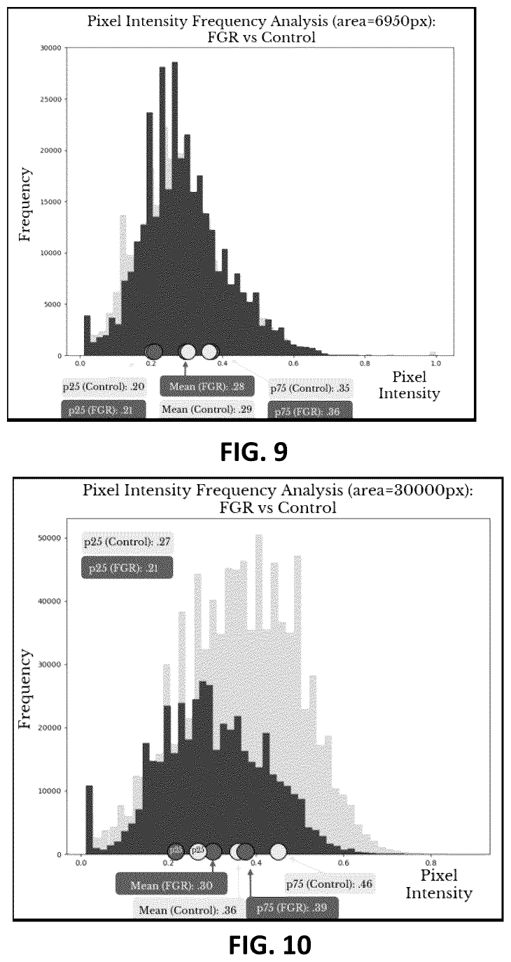

Plotted pixel intensity frequencies for FGR and Control patients in a defined area of 6950 pixels, revealed similar distributions between the groups (FIG. 9). At the 25th percentile, Control and FGR patients have normalized pixel intensities of 0.20 and 0.21, respectively. The mean pixel intensities are closely matched, with Control at 0.29 and FGR at 0.28. Similarly, the 75th percentile shows pixel intensities of 0.35 for Control and 0.36 for FGR, further demonstrating the comparability of pixel intensity distributions within this area. When the area increased to 30,000 pixels, the distribution differences between the FGR and Control image datasets became more pronounced compared to the distributions at 6950 pixels (FIG. 10). At the 25th percentile, FGR images presented a normalized pixel intensity of 0.21, noticeably lower than the Control's 0.27. The mean pixel intensity for FGR was 0.30, in contrast to the Control's 0.36. Furthermore, at the 75th percentile, FGR's pixel intensity was 0.39, significantly below the Control's 0.46, highlighting the increased differentiation in pixel intensity distribution between the two groups at this larger area.

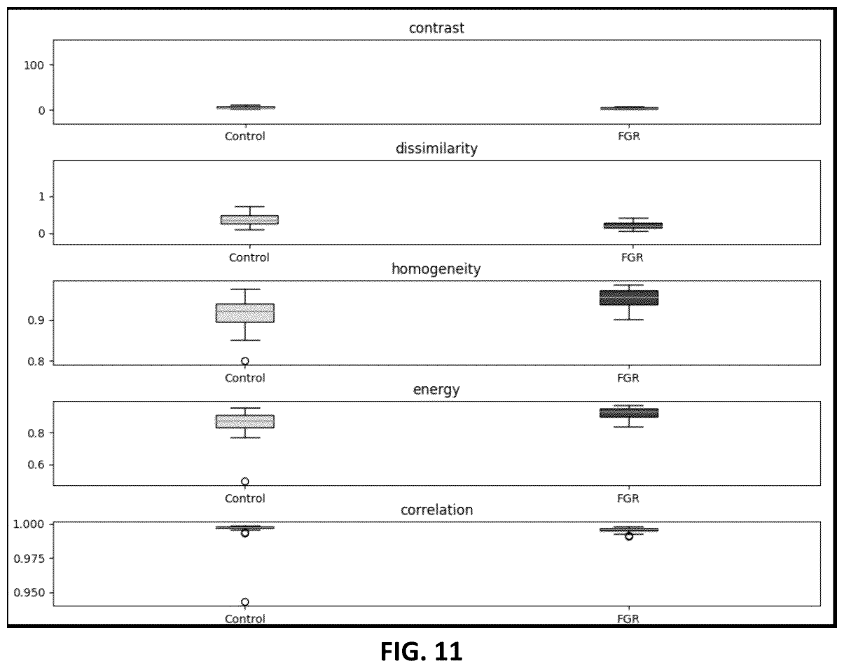

Using the Gray Level Co-occurrence Matrix (GLCM) method, significant distinctions between the Control and Fetal Growth Restriction (FGR) groups were observed (FIG. 11). The GLCM features, including contrast, dissimilarity, homogeneity, energy, and correlation, offer insight into the texture characteristics of the images. For the Control group, the mean contrast value was 9.20, with the interquartile range (IQR) ranging from 3.58 to 7.58, indicating a higher variability in pixel intensity differences compared to the FGR group, which had a lower mean contrast of 3.66 and a narrower IQR of 2.23 to 5.23.

Dissimilarity measures also presented differences; the Control group's mean was 0.42 with an IQR of 0.27 to 0.48, compared to the FGR group's mean of 0.21 and IQR of 0.14 to 0.28, further indicating that the Control group's images have more variation in their pixel values.

Homogeneity and energy, which indicate the uniformity and textural regularity of the images, were higher on average in the FGR group (means of 0.95 and 0.92, respectively) than in the Control group (means of 0.92 and 0.86, respectively). This suggests that the FGR group's images are generally more uniform and consistent in texture compared to those of the Control group.

Finally, correlation, a measure of how predictably one pixel's intensity is related to its neighbor, showed perfect or near-perfect scores (mean of 1.00) in both groups, indicating a high degree of predictability in pixel intensity relationships across both conditions.

### Example 2—Digital Biomarkers for Fetal Growth Restriction Based on Multi-Scale, Multiphysics Modeling and Machine Learning

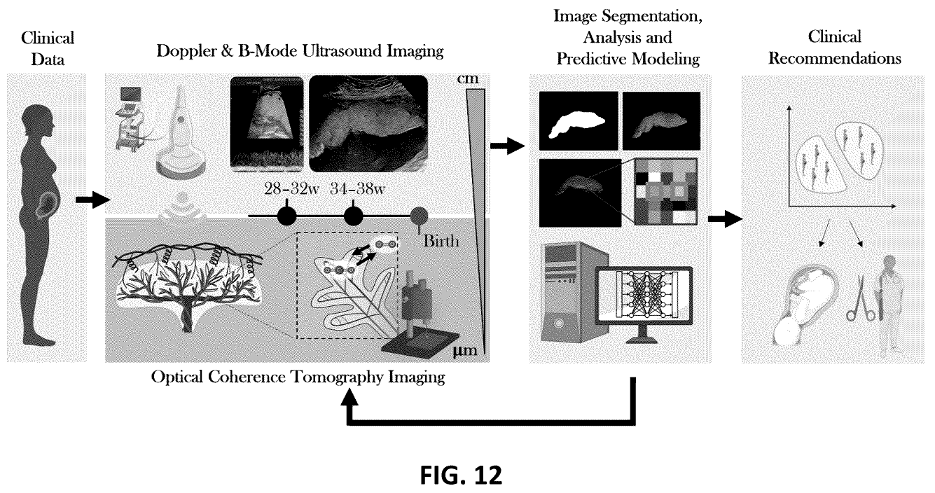

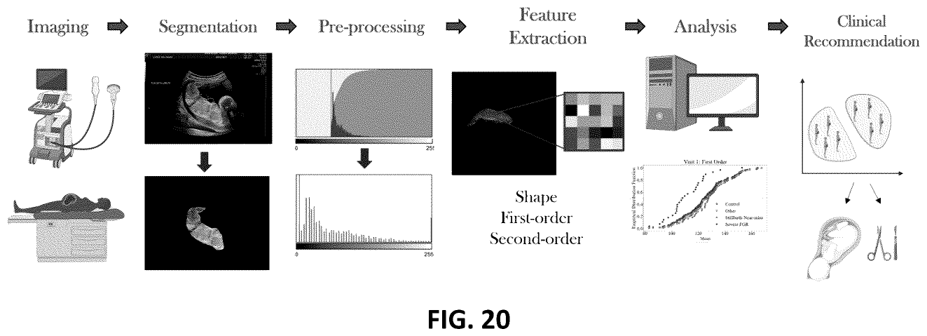

In Example 2, a digital biomarker project workflow is described for fetal growth restriction (FGR) (FIGS. 12 and 20). This includes starting with current clinical data, performing Doppler and B-Mode ultrasound imaging, performing optical coherence tomography, performing subsequent image segmentation, analysis, and predictive modeling, and providing subsequent clinical recommendations.

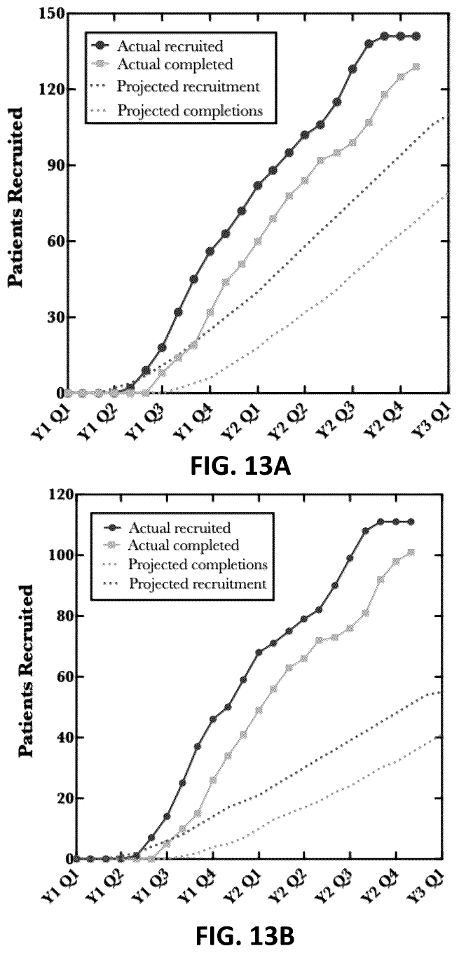

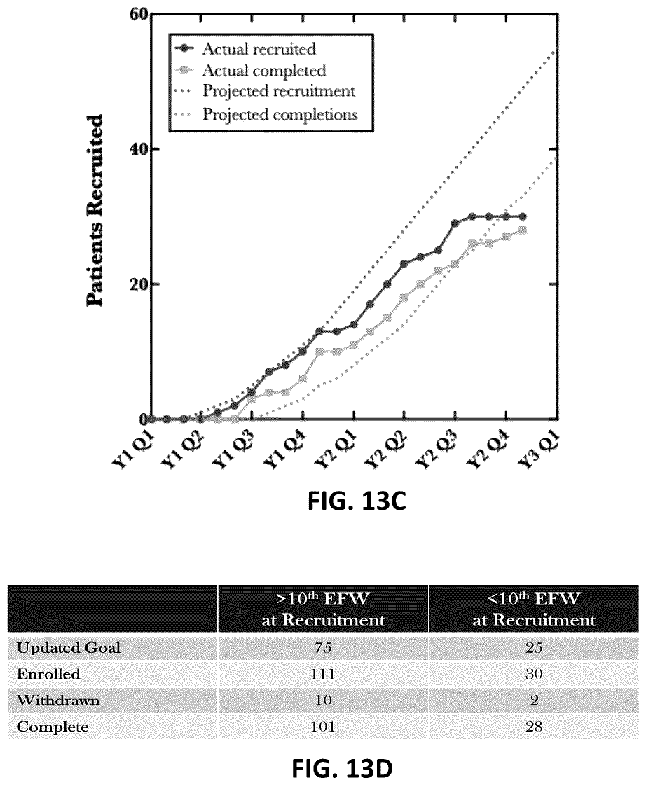

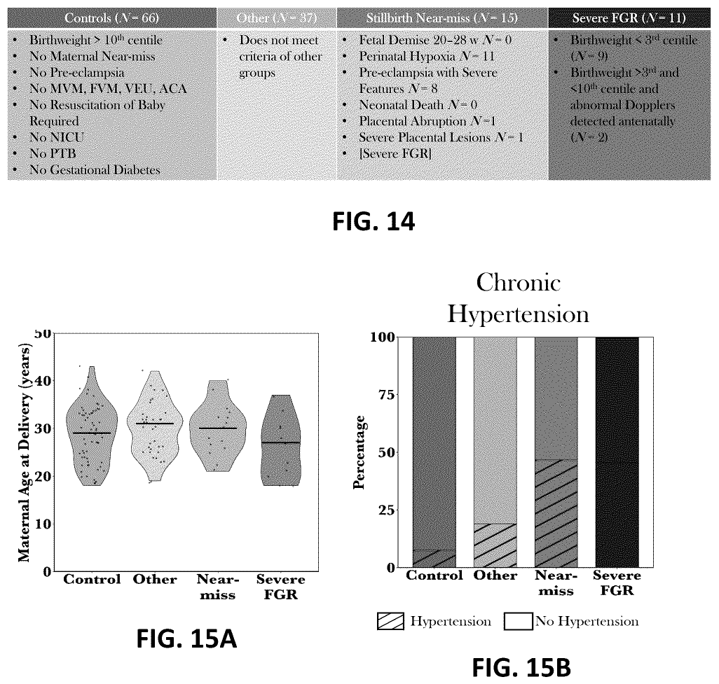

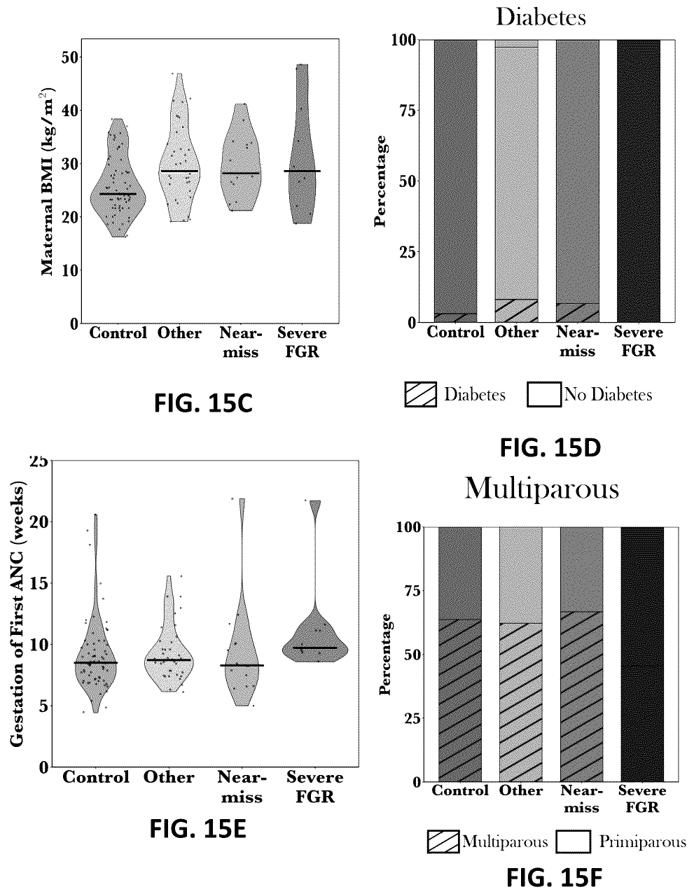

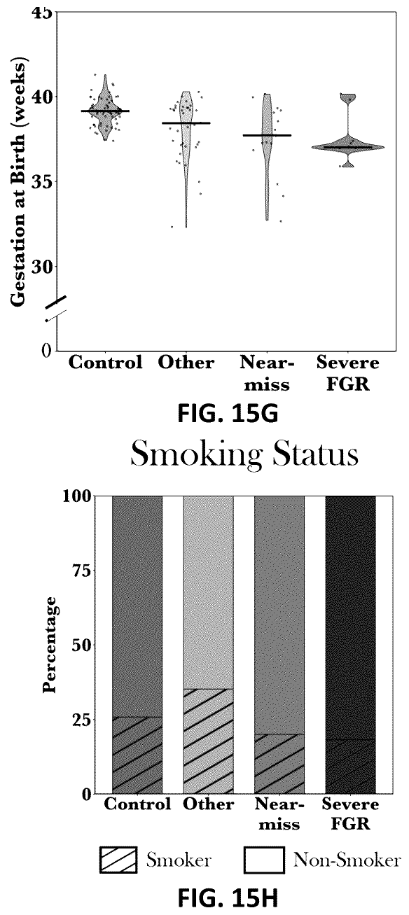

Patients were recruited for the studies of Example 2 (FIG. 13), and the population is described in FIG. 14 (N=122 Patients; 64 Control, 34 Other, 14 Stillbirth Near-miss, 10 Severe FGR). Maternal clinical data, including maternal age at delivery (FIG. 15A), maternal BMI (FIG. 15C), gestation time of first ANC (FIG. 15E), and gestation time and birth (FIG. 15G), was collected, and the patients were stratified in control, other, near-miss, and severe FGR groups. Patients with chronic hypertension (FIG. 15B), pre-pregnancy diabetes (FIG. 15D), multiparous properties (FIG. 15F), and those that smoke (FIG. 15H) were also identified.

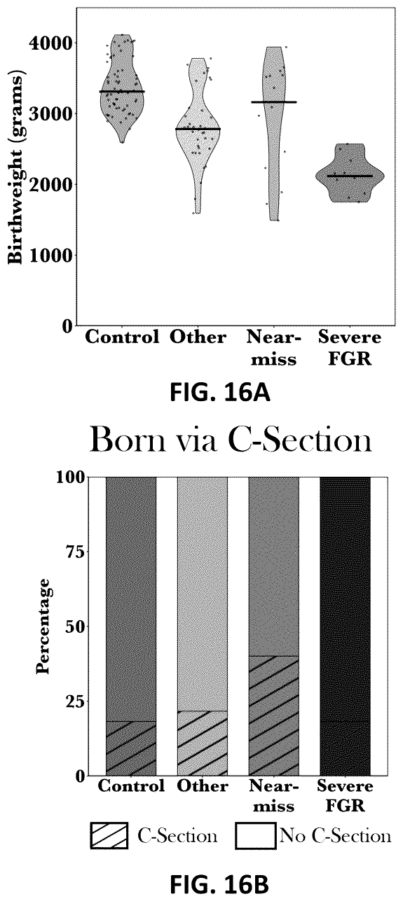

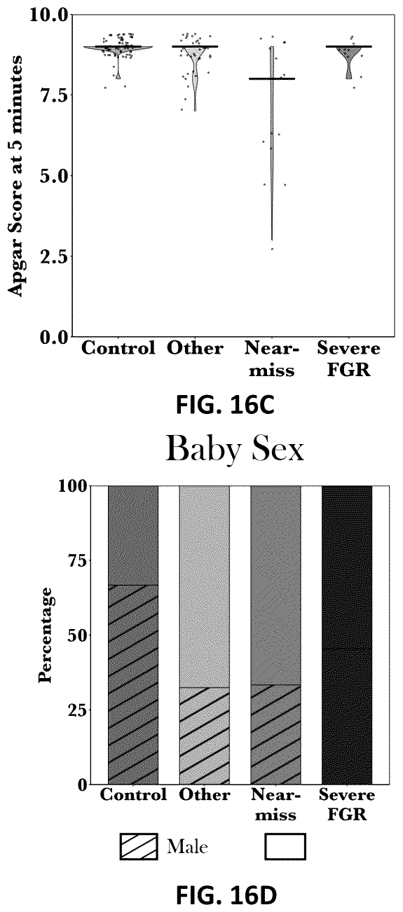

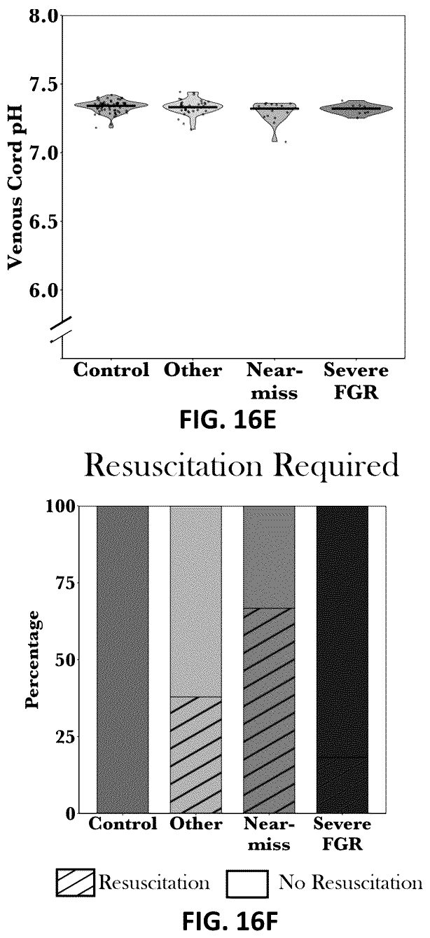

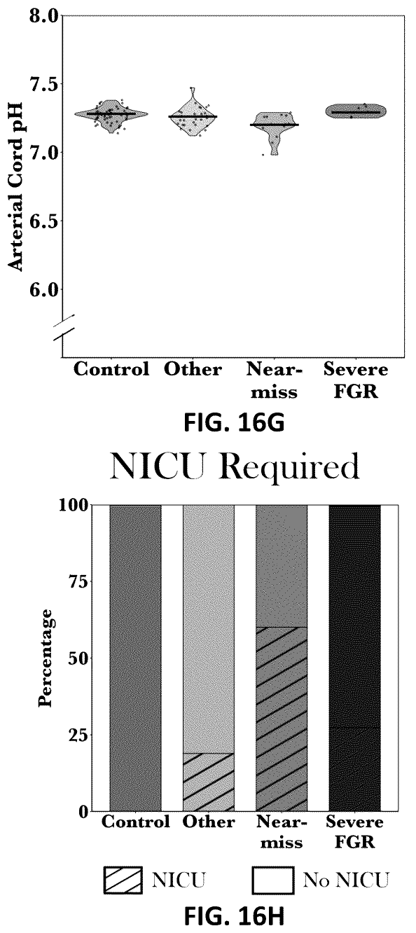

Baby clinical data, including birthweight (FIG. 16A), Apgar score at 5 minutes (FIG. 16C), venous cord pH (FIG. 16E), and arterial cord pH (FIG. 16G), were collected. Babies born via C-section (FIG. 16B), baby sex (FIG. 16D), babies that needed resuscitation (FIG. 16F), and babies that required NICU (FIG. 16H), were also identified.

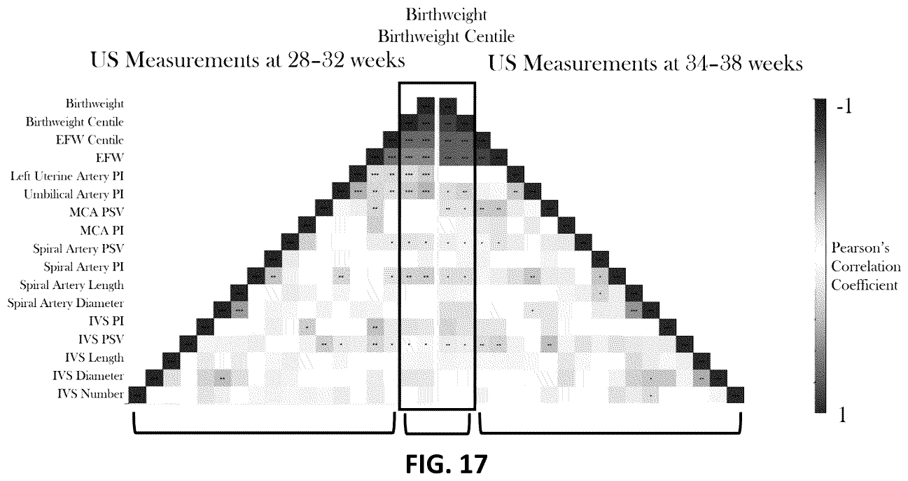

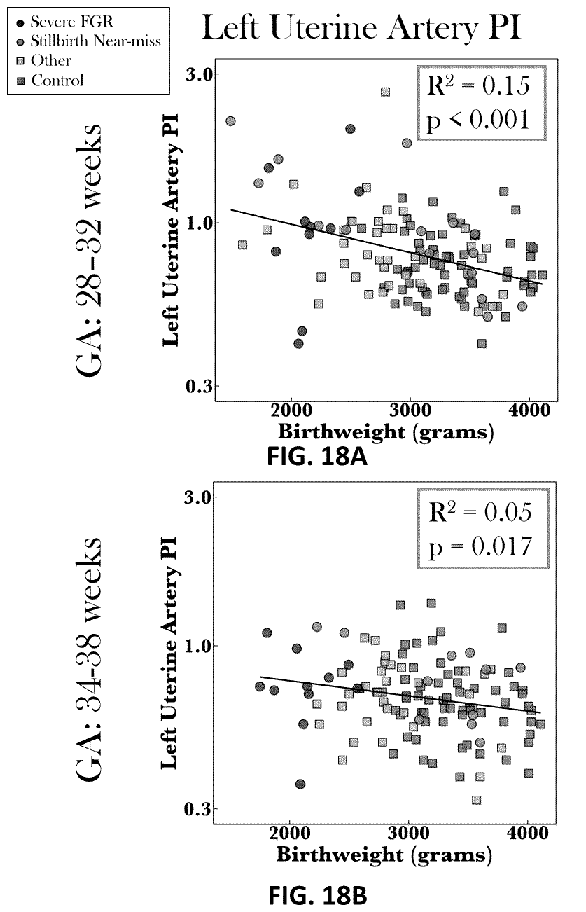

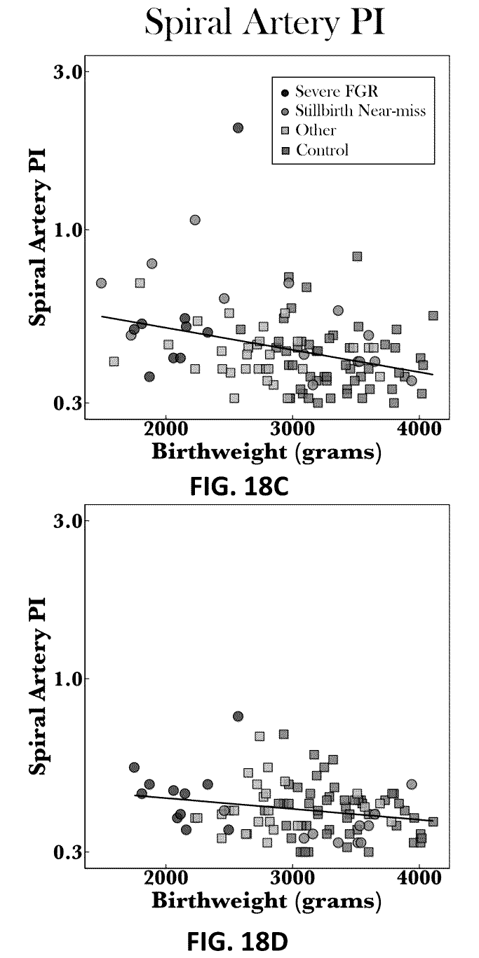

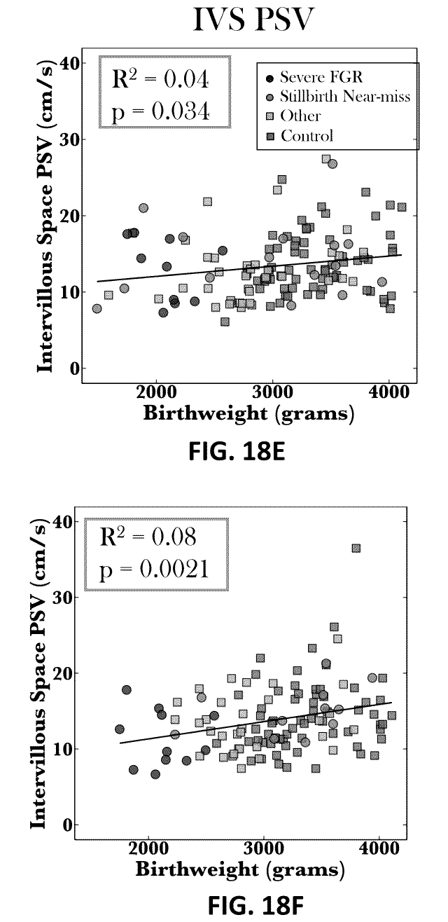

Doppler ultrasound measurements were performed on pregnant women at 2 time points, 28-32 weeks and 34-38 weeks (FIG. 17). It is shown that these Doppler ultrasound measurements correlate with birthweight (FIG. 18A-F).

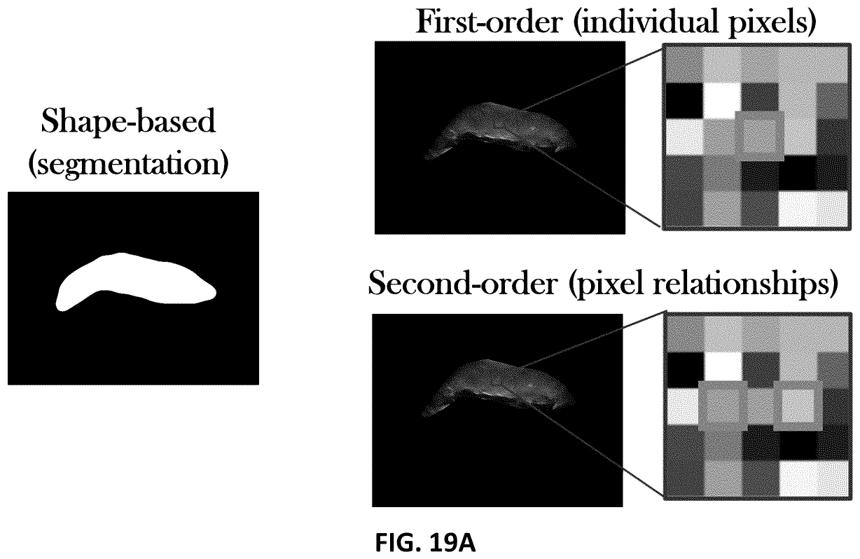

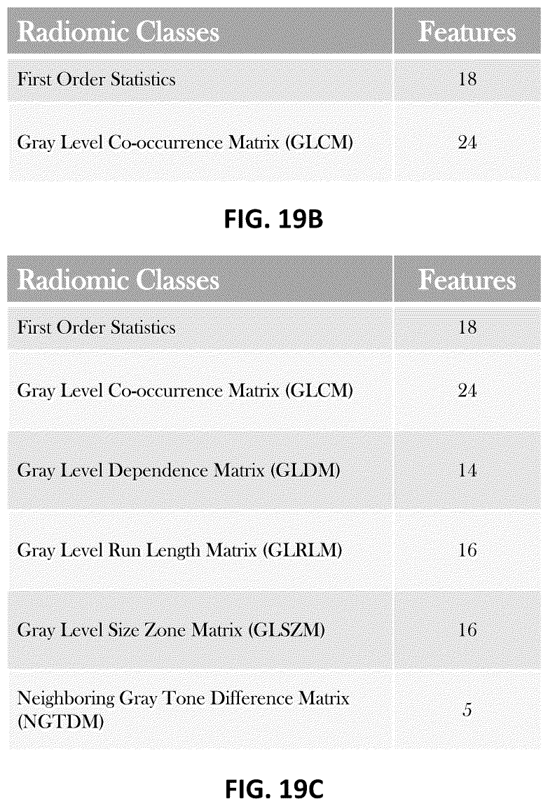

Image texture analysis was performed on the ultrasound images using Py-Radiomics, an open-source python package for extraction of Radiomic features from medical images (FIG. 19A-C). The majority of output features comply with Imaging Biomarker Standardization Initiative (ISBI) definitions.

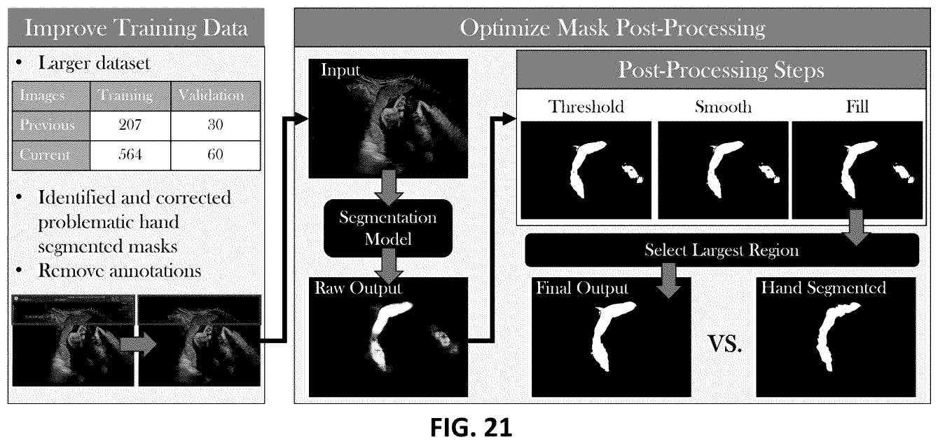

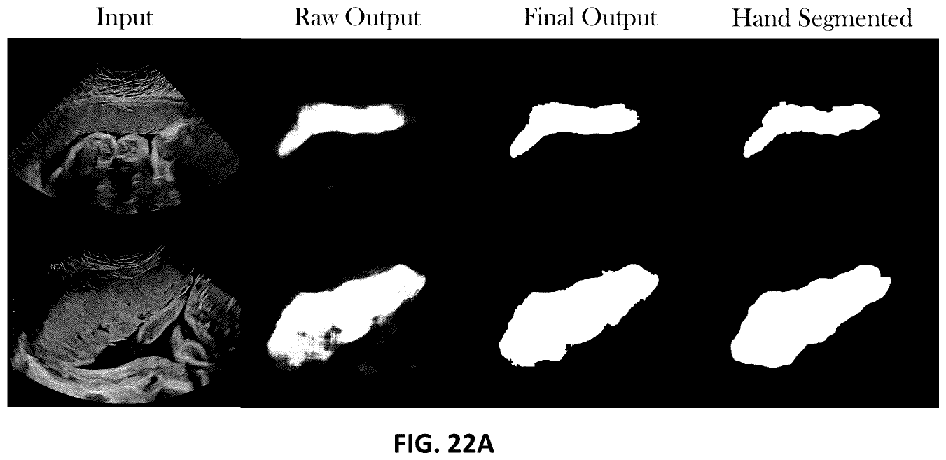

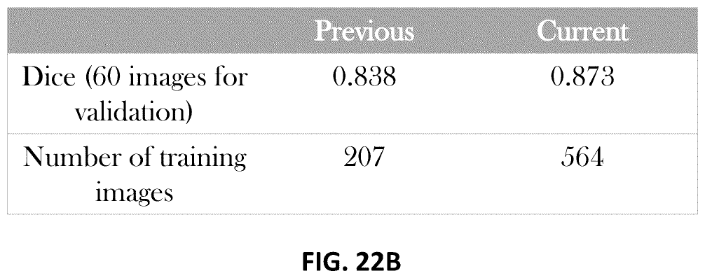

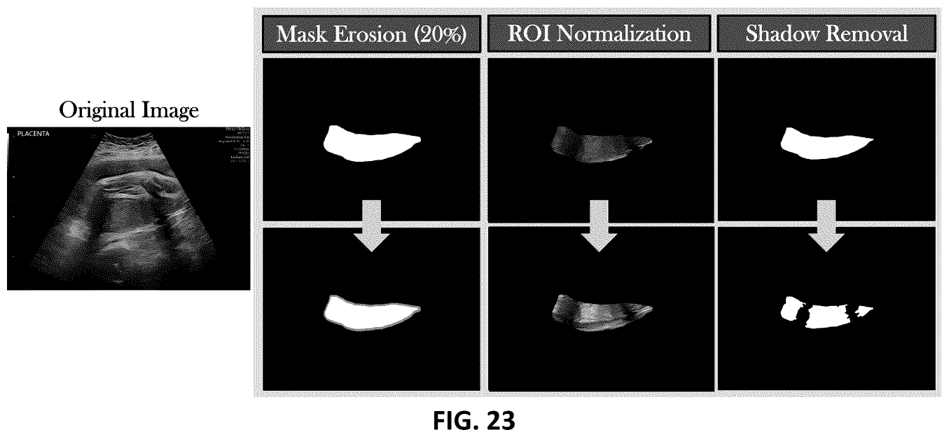

Image segmentation was performed (FIG. 21, 22A, and 22B), wherein training data is improved with larger datasets. Problematic hand segmented masks were identified and corrected, and annotations were removed. Mask post-processing was optimized, wherein the ultrasound images are inputted into a segmentation model, providing a raw output. Then post- processing thresholding, smoothing, and filling is performed, and the largest region is selected. Image and mask preprocessing, which includes mask erosion, ROI normalization, and shadow removal was also performed (FIG. 23)

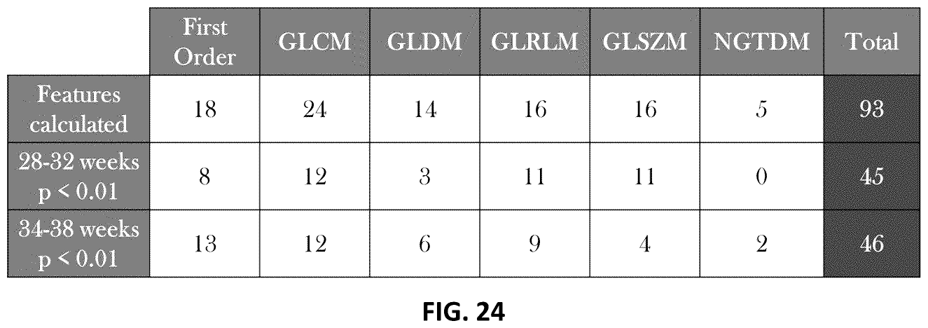

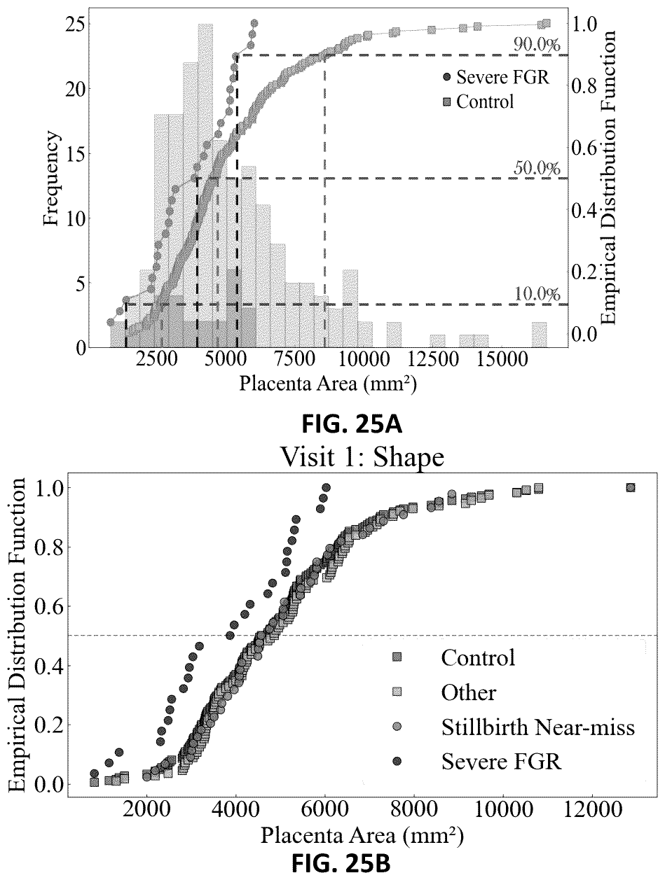

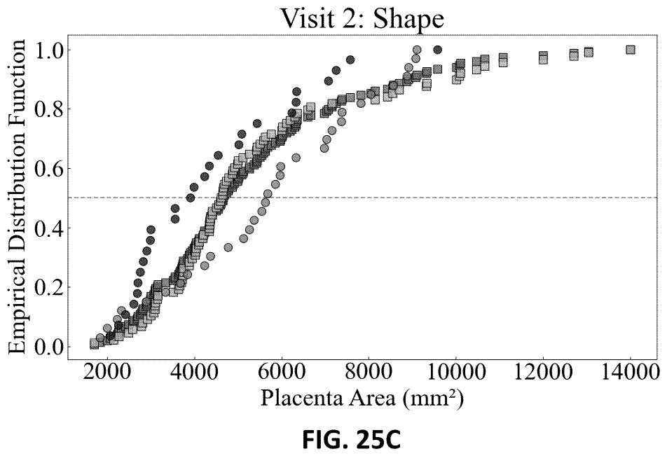

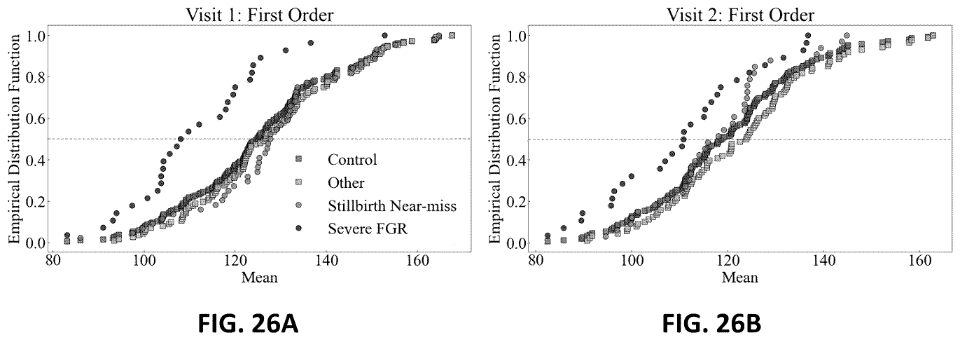

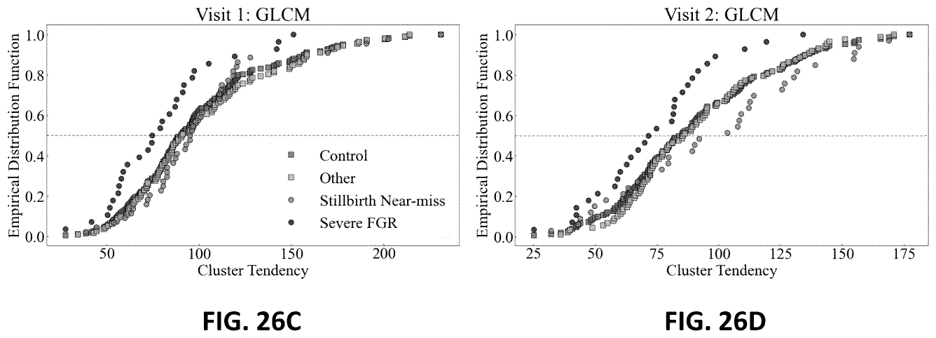

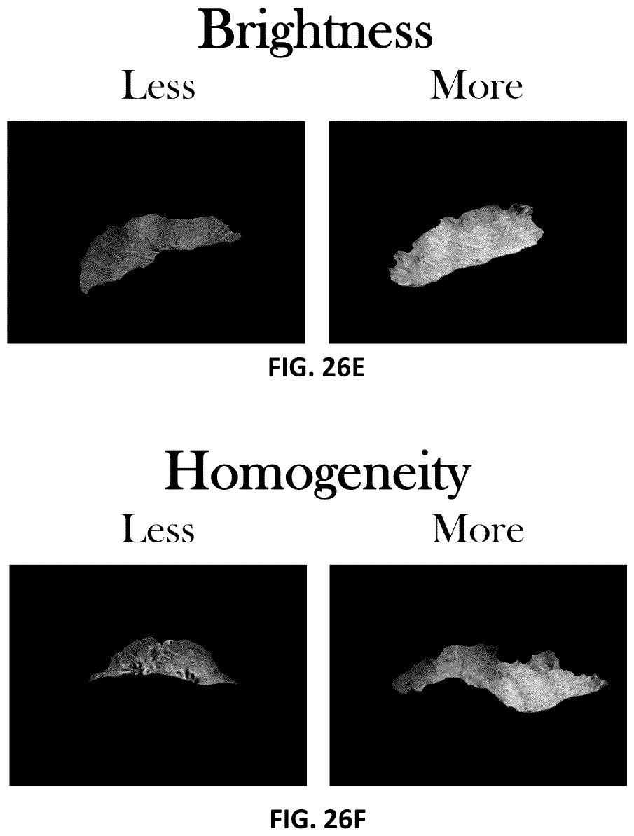

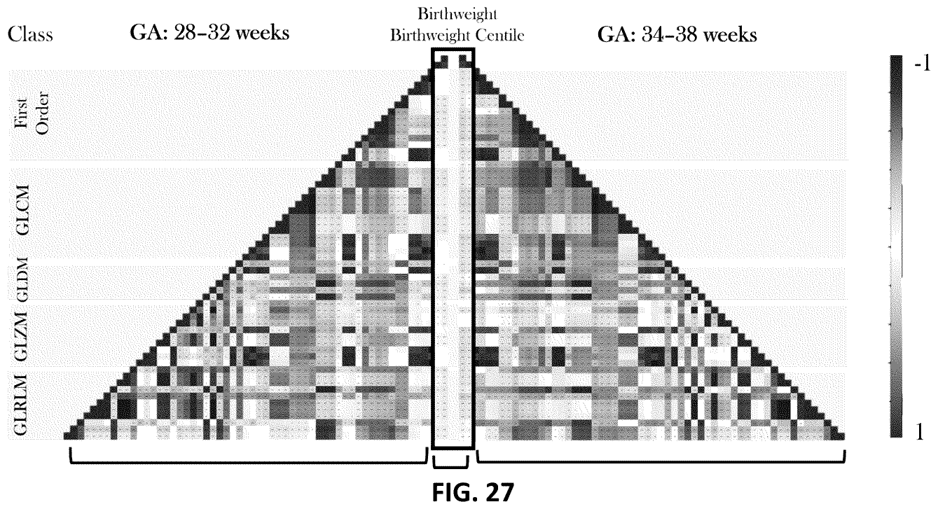

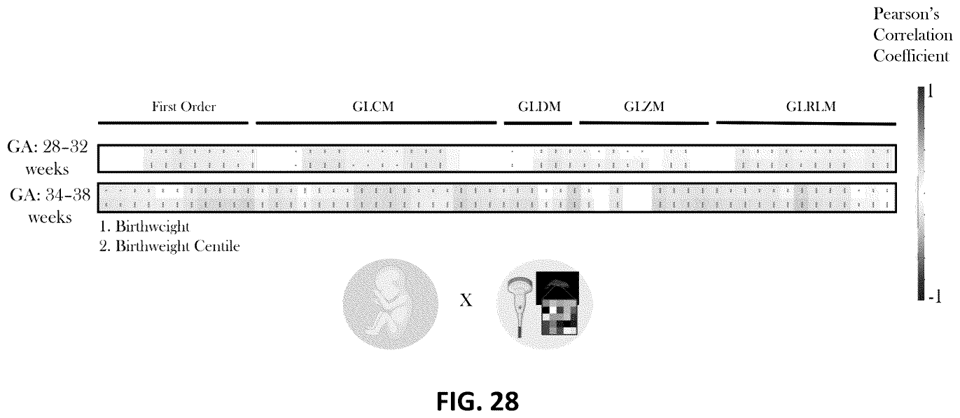

Images Texture image analysis comparing severe FGR, stillbirth near-miss, other, and control groups was performed (FIG. 24), and differences in the empirical distribution function (EDF) of each group were observed (FIG. 25A-C). Cluster Tendency is a measure of groupings of voxels with similar gray-level values. It was found that control placentas are more homogenous than FGR placentas. Examples of texture image analysis can be seen in FIG. 26A-D, where more and less bright images (FIG. 26E) as well as more or less homogeneous images (FIG. 26F) were identified. It was found that texture analysis features correlate with birthweight (FIGS. 27 and 28), with the strongest correlations being with birthweight/birth centile.

The texture analysis yielded several different texture parameters characterizing patterns of gray level intensities within the images. GLDM Dependence Non Uniformity measures the similarity of dependence throughout the image, with a lower value indicating more homogeneity among dependencies in the image. GLDM Gray Level Non Uniformity measures the similarity of gray-level intensity values in the image, where a lower GLN value correlates with a greater similarity in intensity values. GLRLM Gray Level Non Uniformity measures the similarity of gray-level intensity values in the image, where a lower GLN value correlates with a greater similarity in intensity values. GLRLM Run Length Non Uniformity measures the similarity of run lengths throughout the image, with a lower value indicating more homogeneity among run lengths in the image. GLSZM Gray Level Non Uniformity measures the variability of gray-level intensity values in the image, with a lower value indicating more homogeneity in intensity values. GLSZM Size Zone Non Uniformity measures the variability of size zone volumes in the image, with a lower value indicating more homogeneity in size zone volumes.

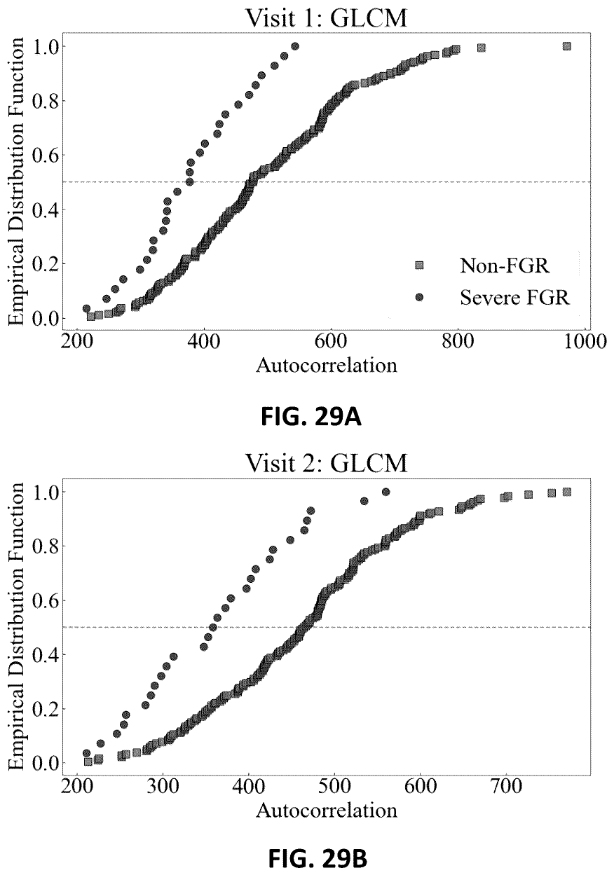

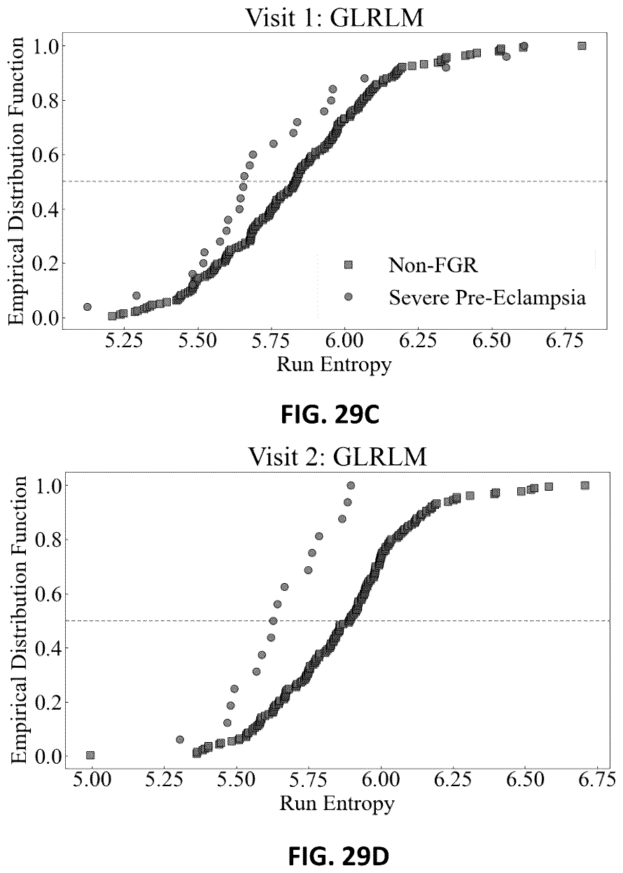

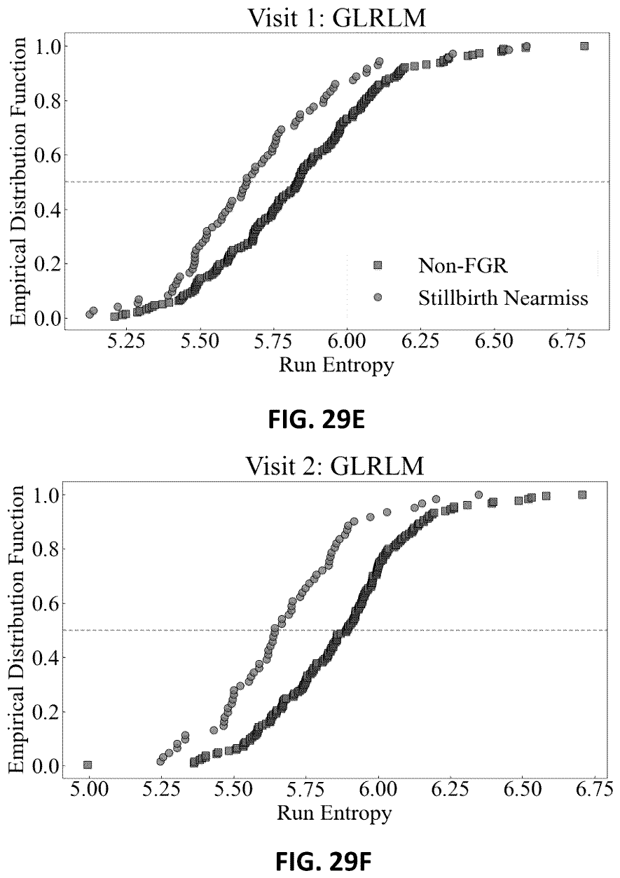

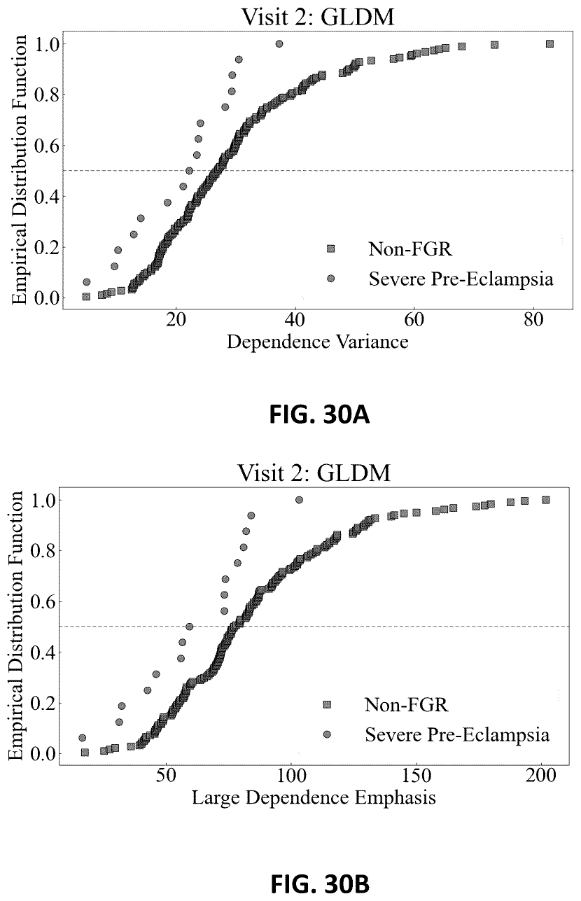

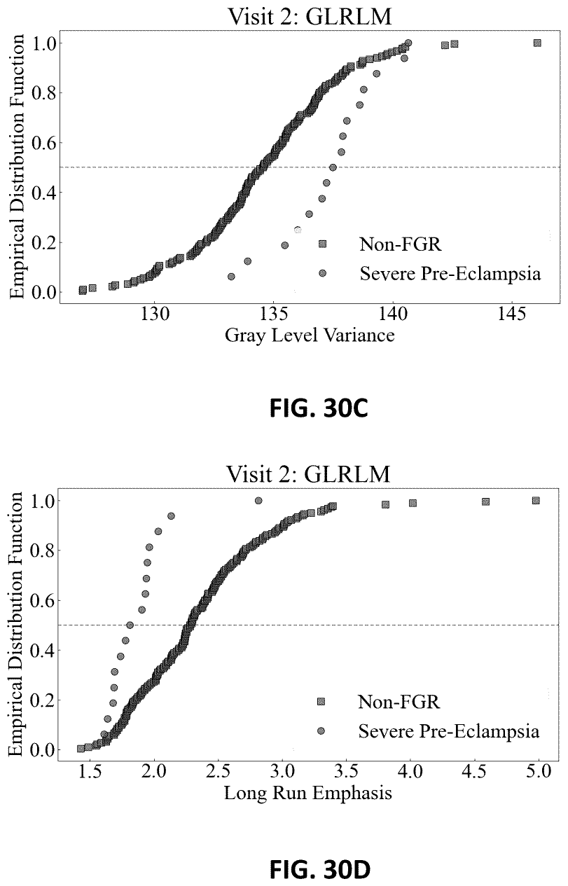

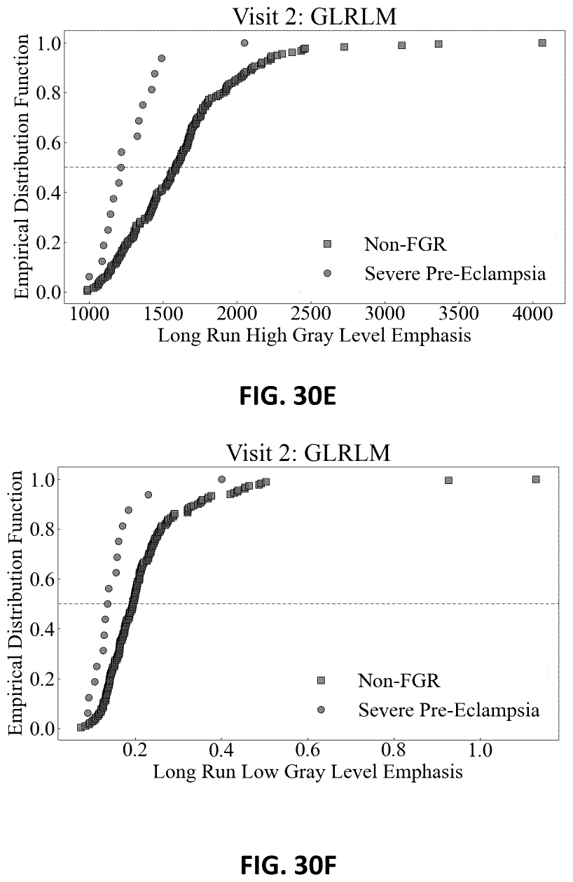

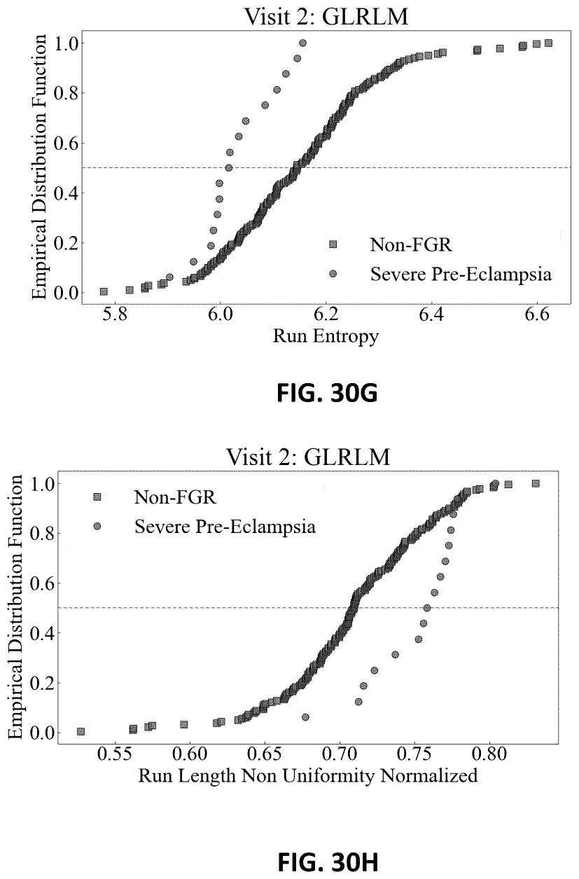

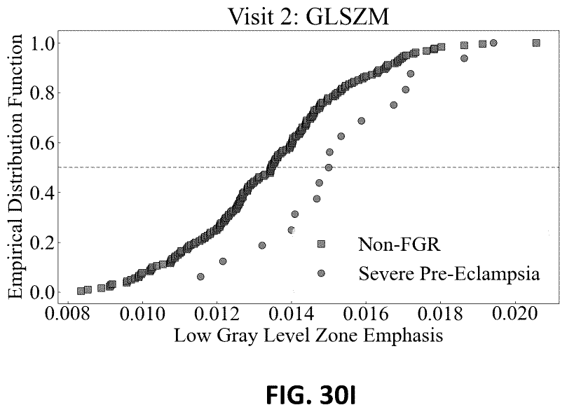

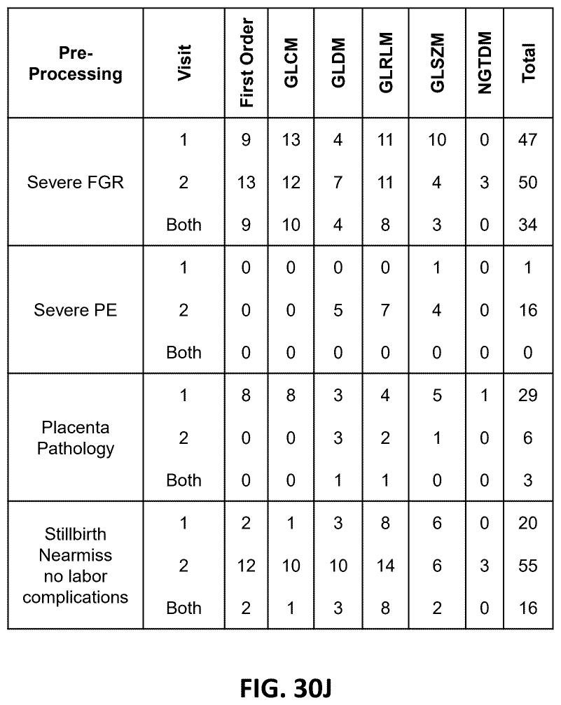

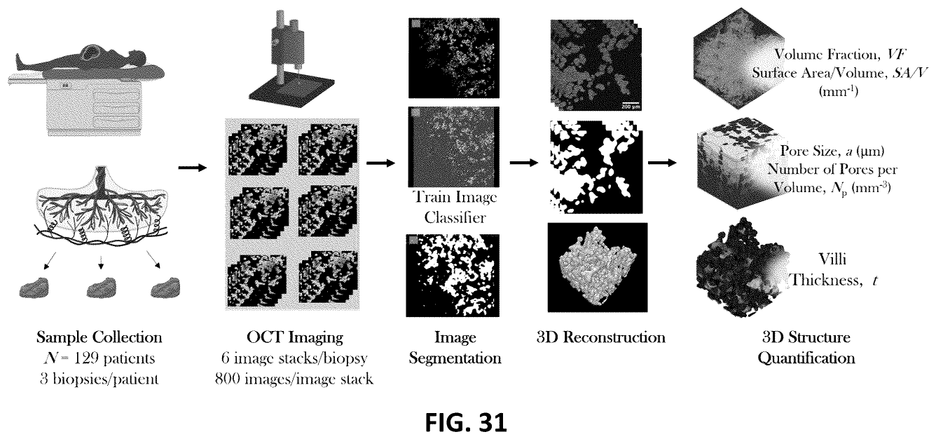

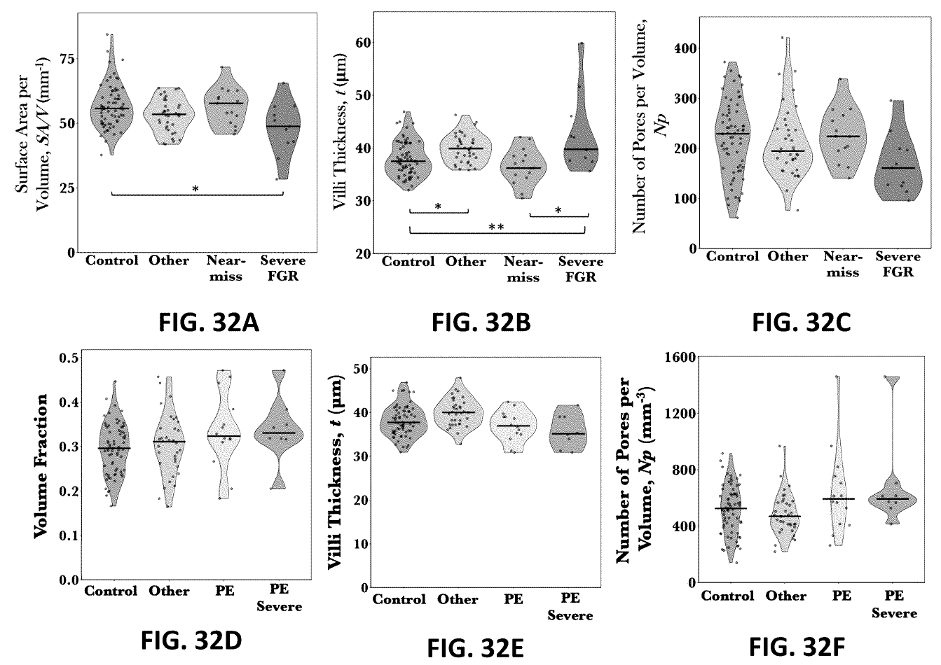

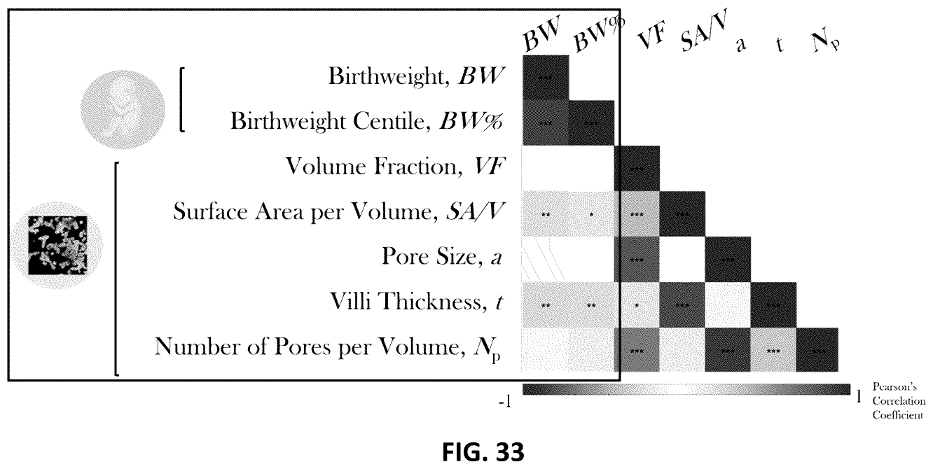

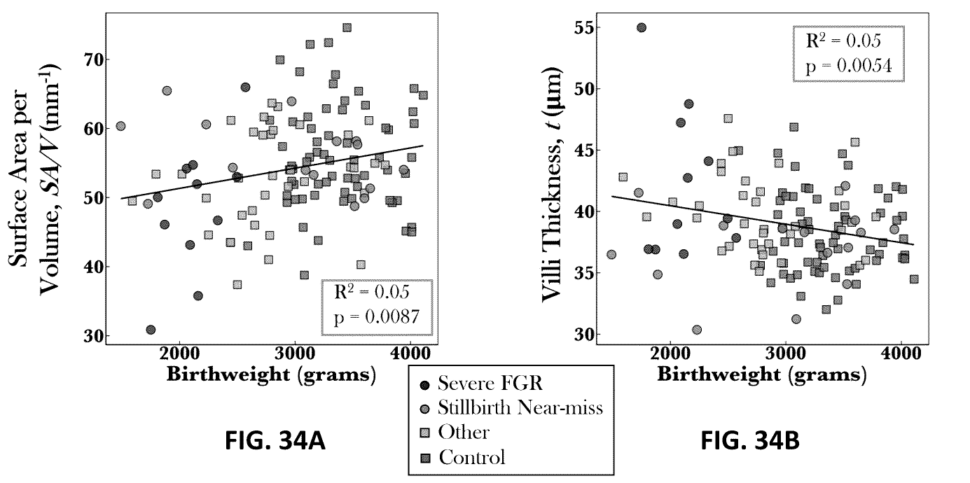

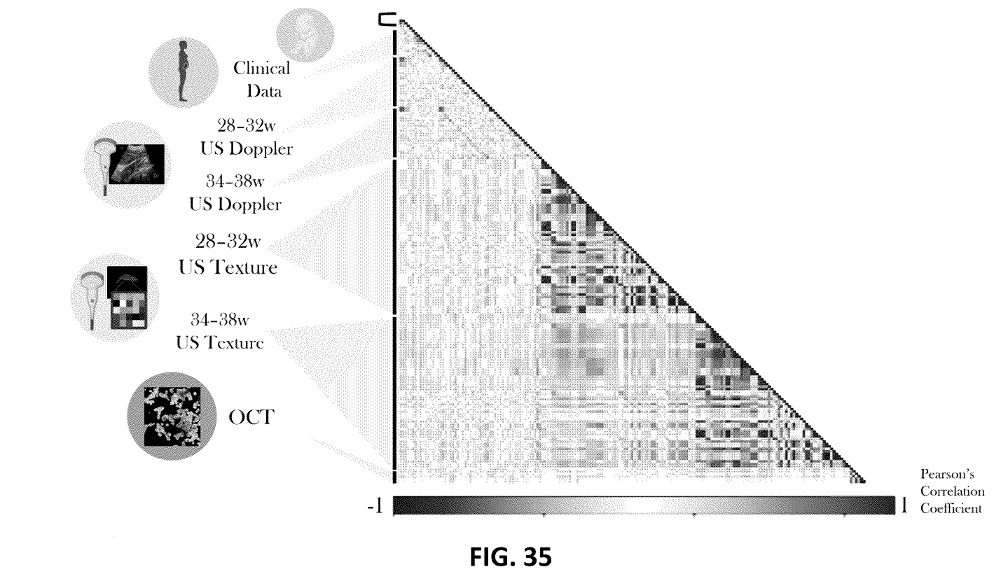

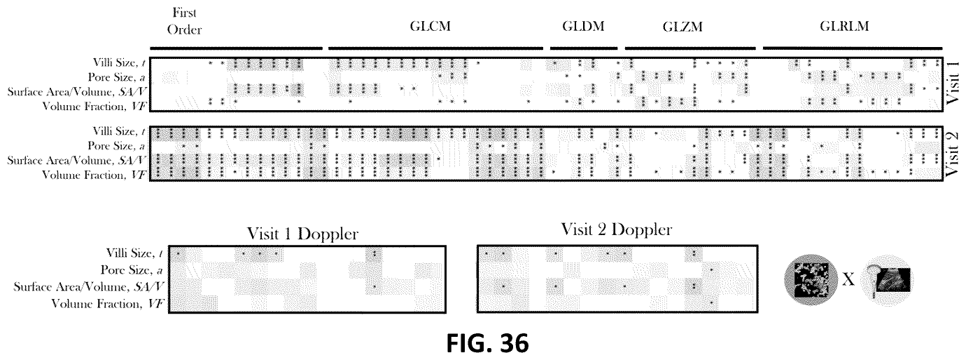

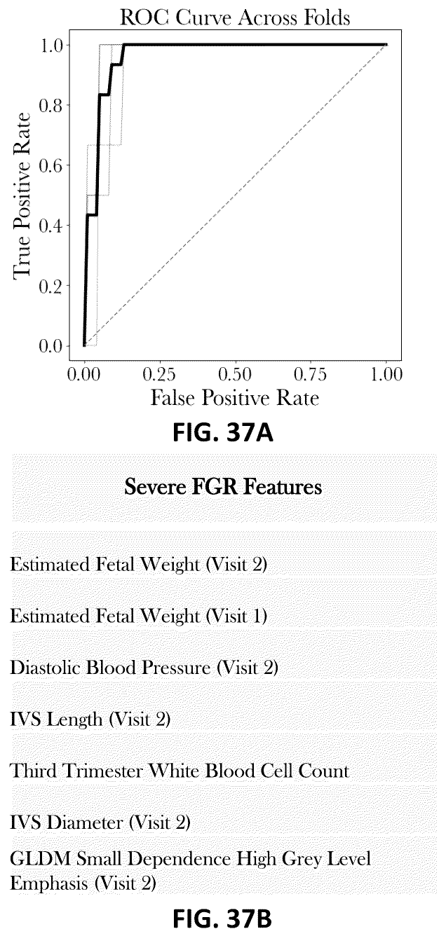

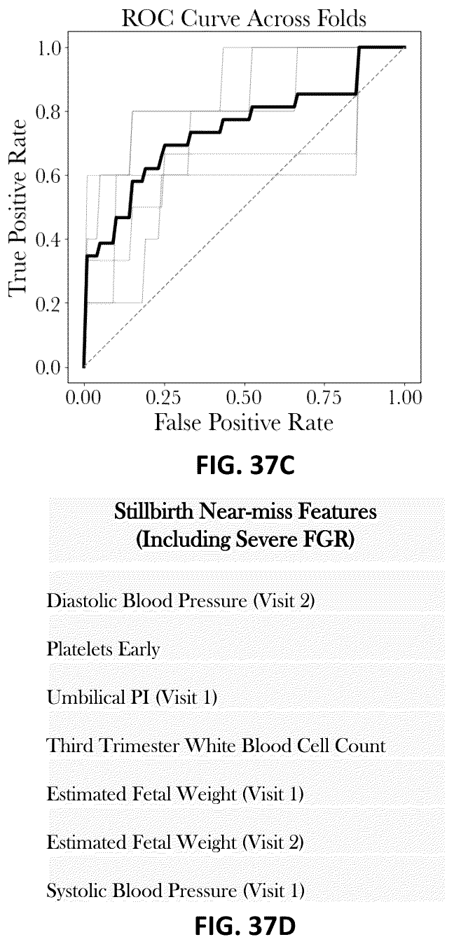

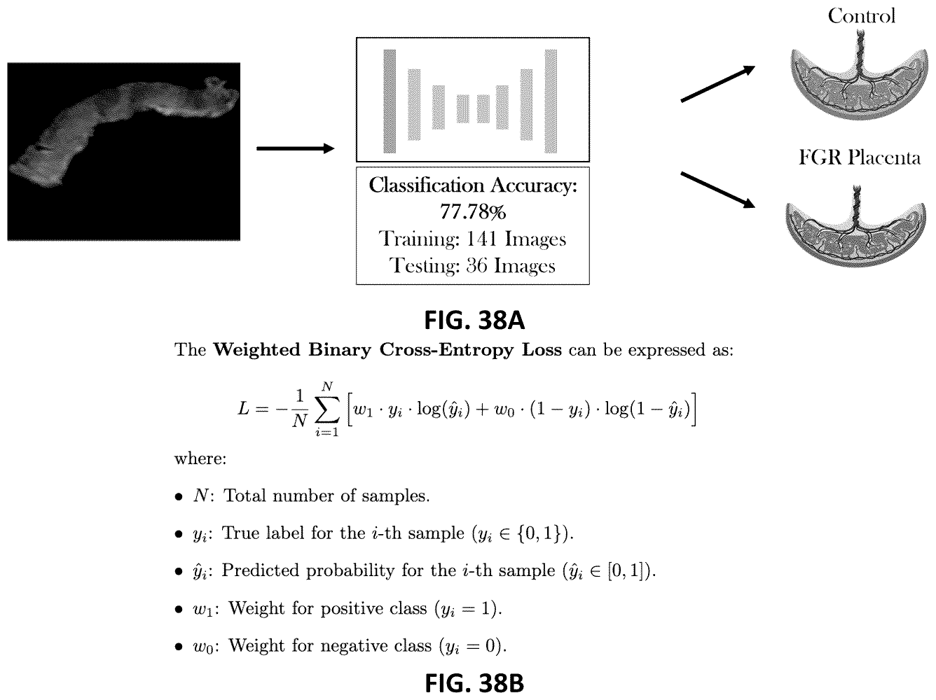

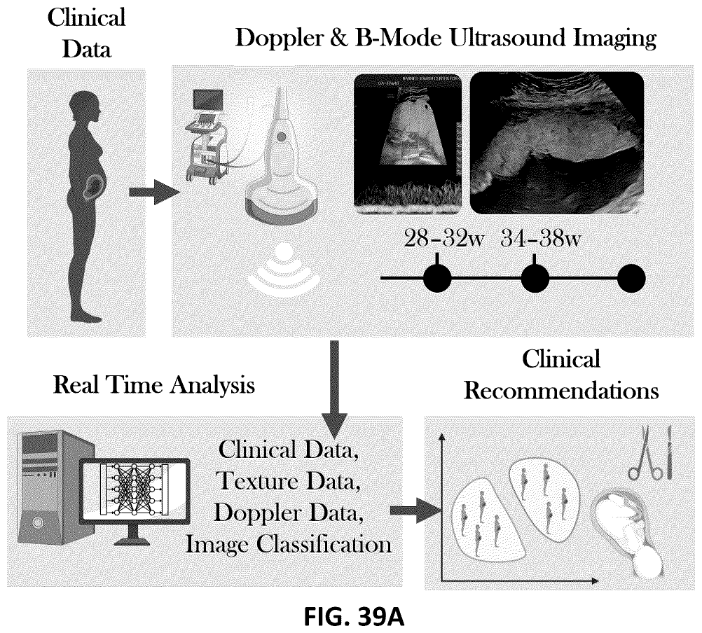

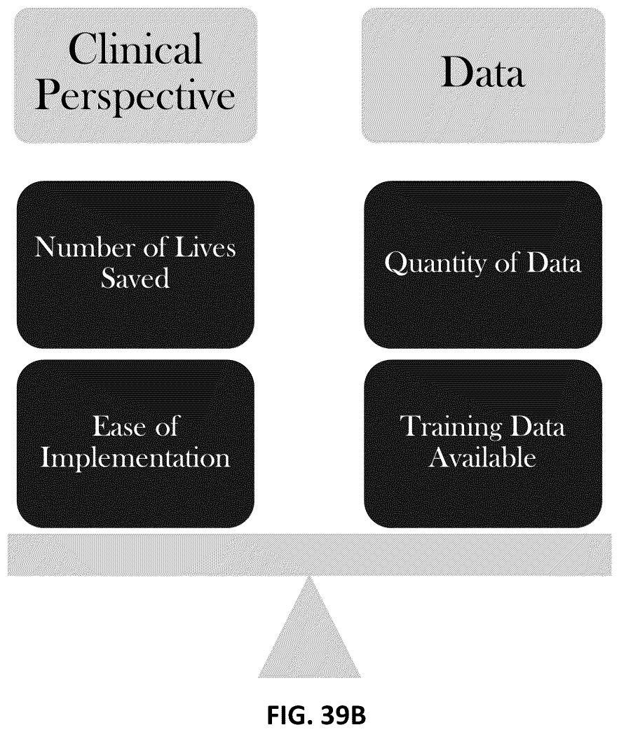

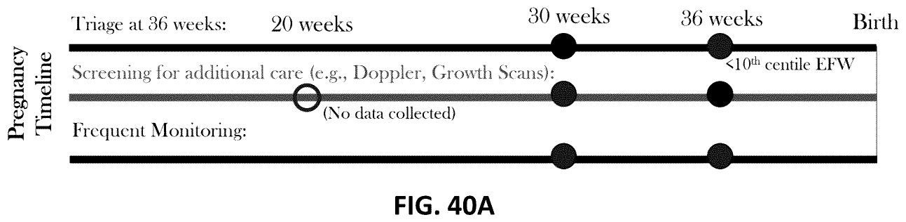

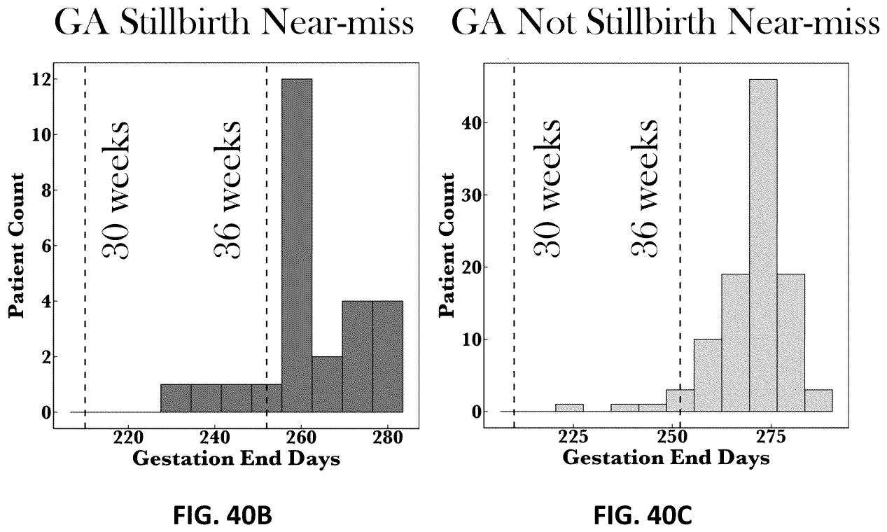

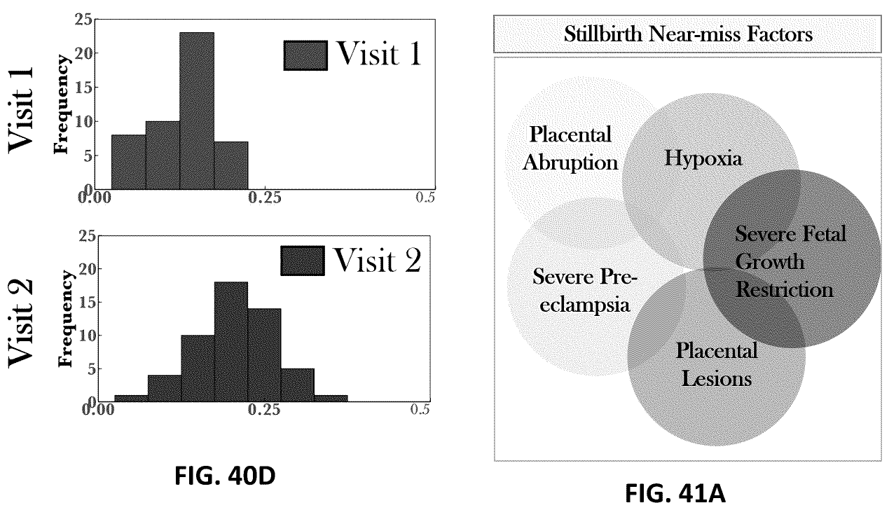

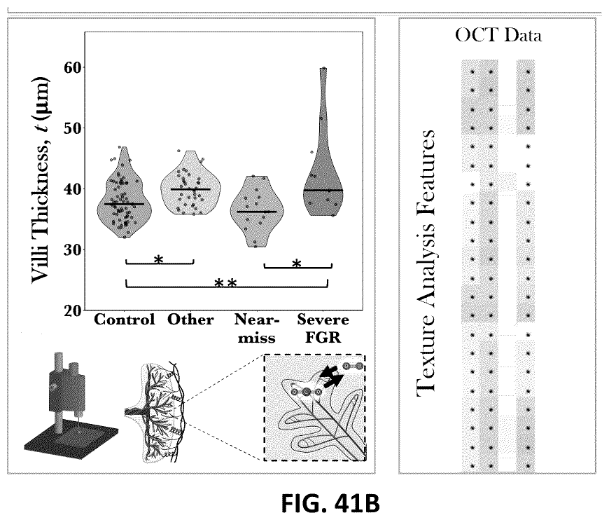

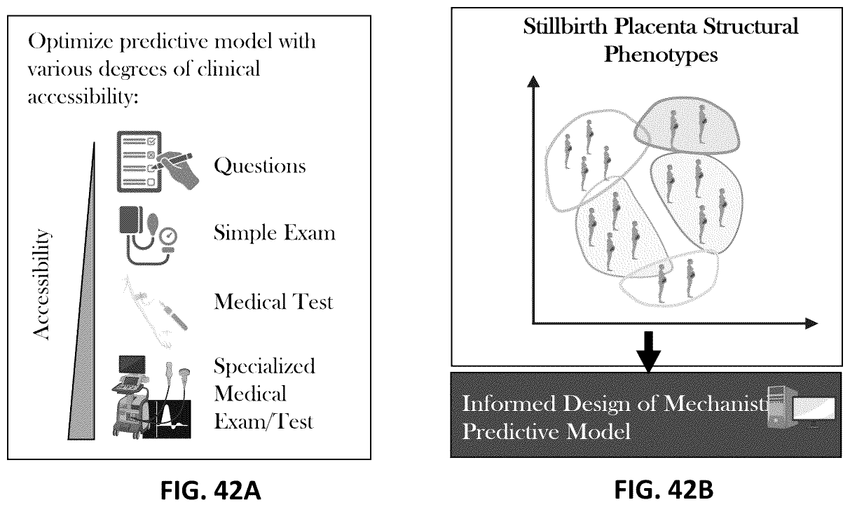

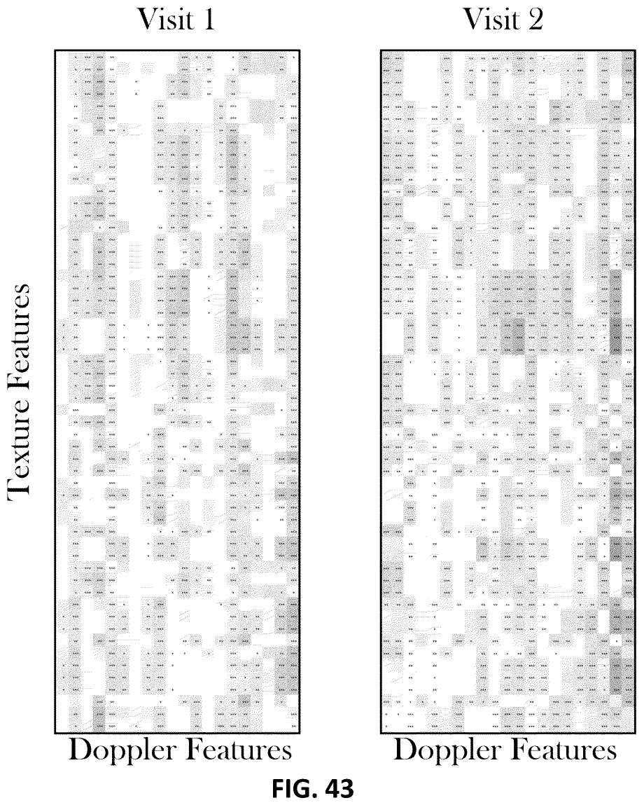

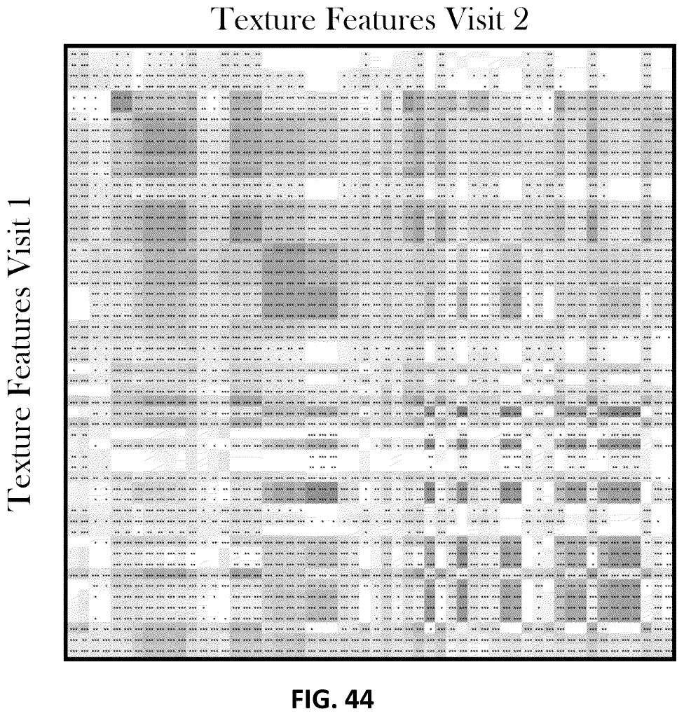

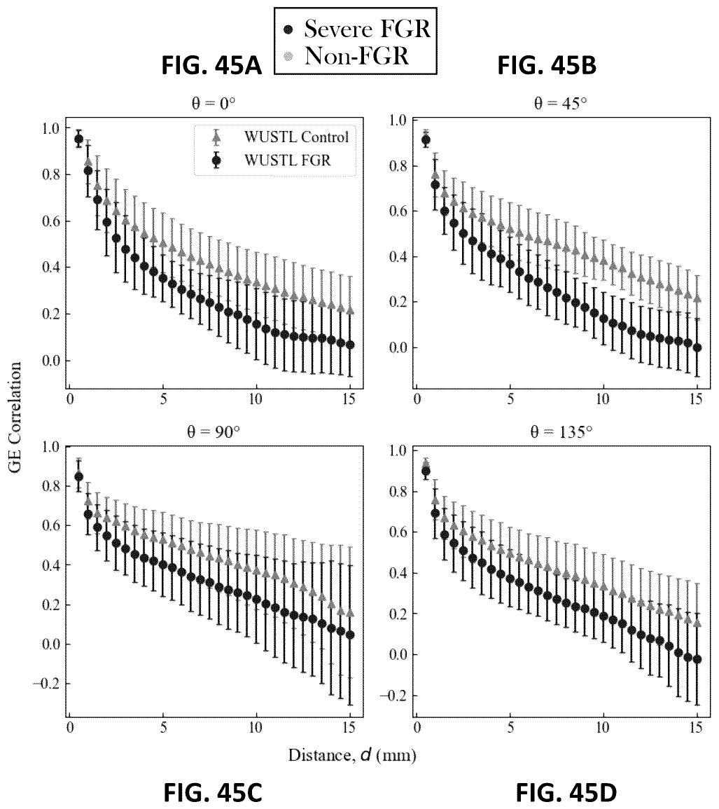

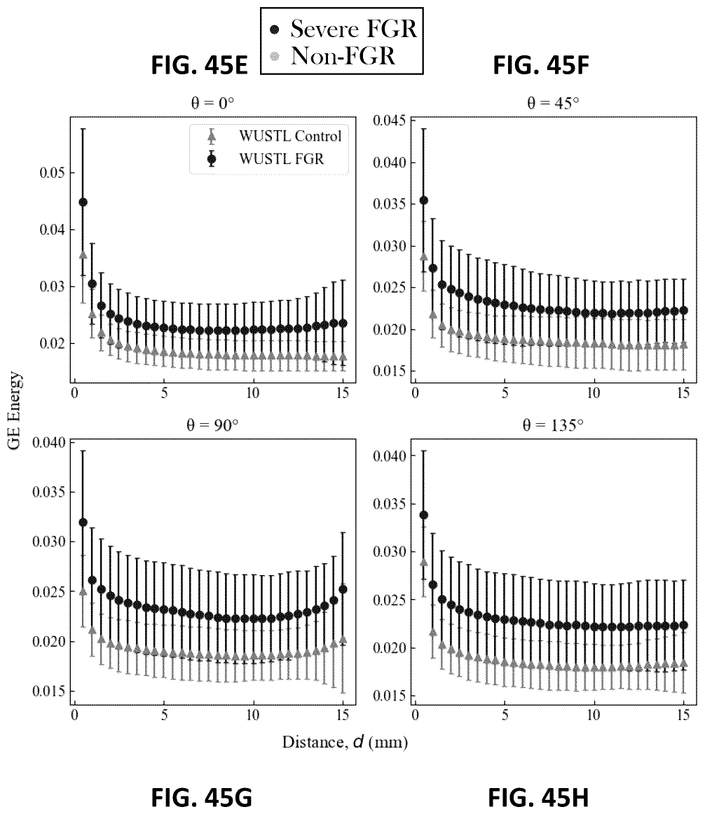

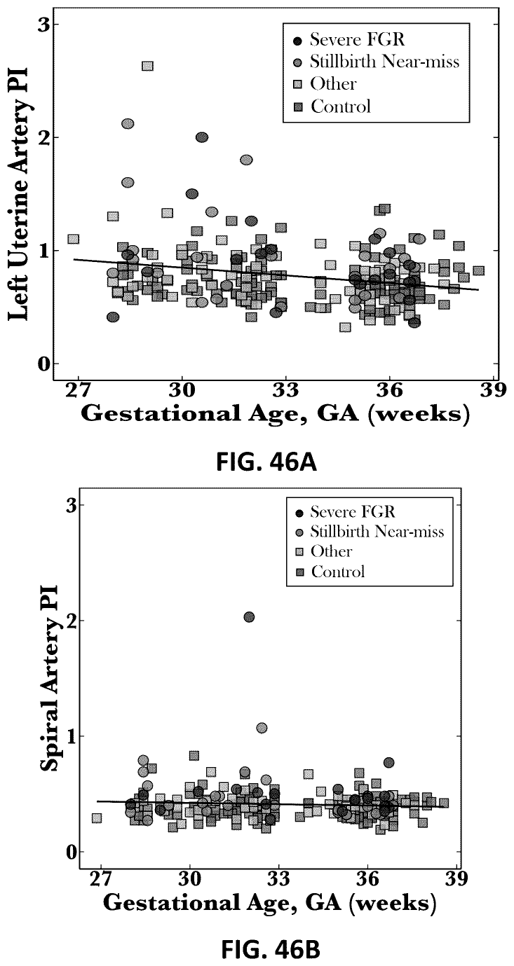

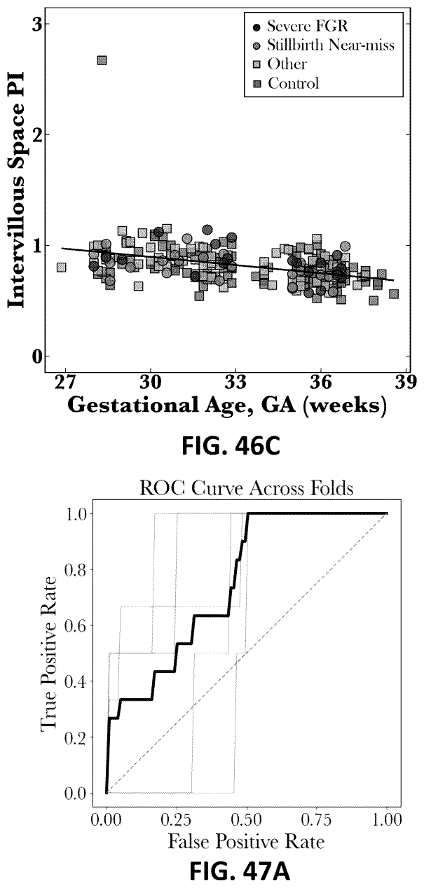

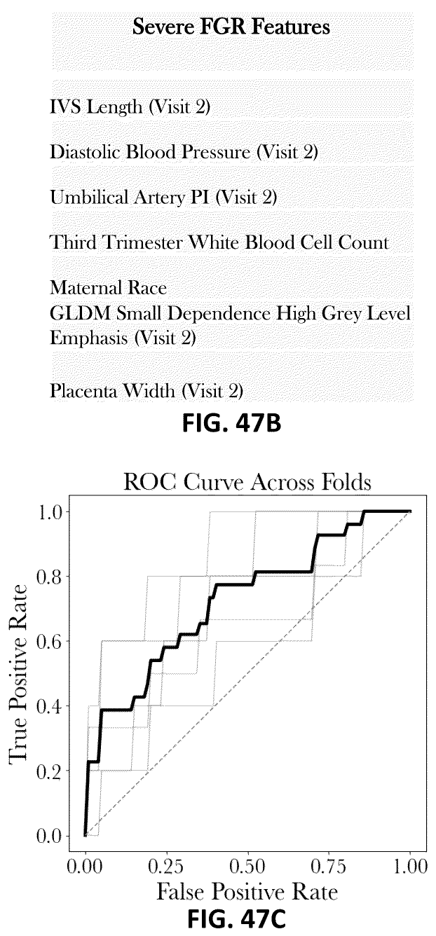

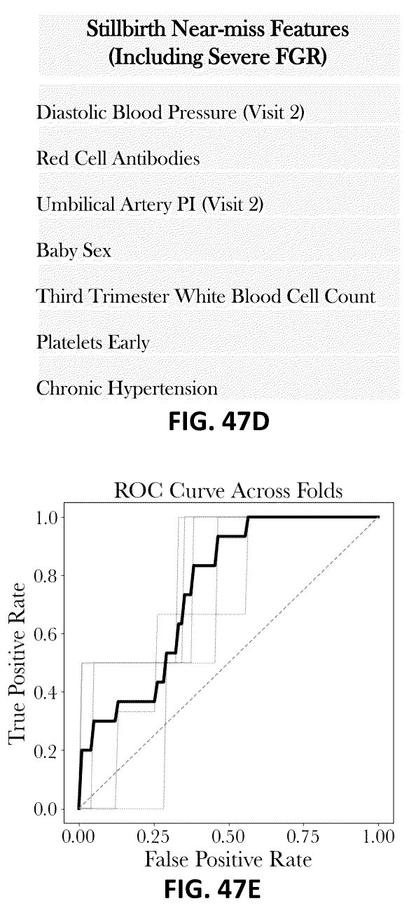

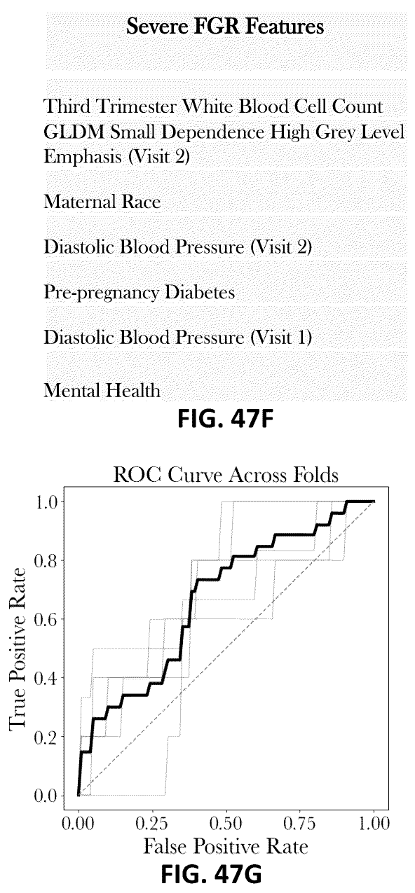

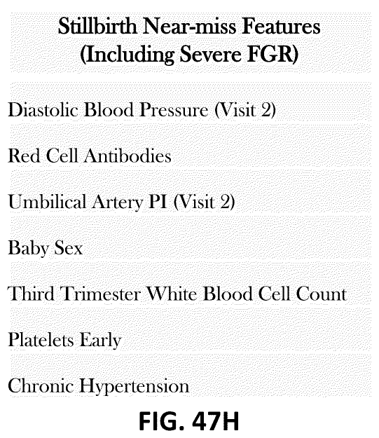

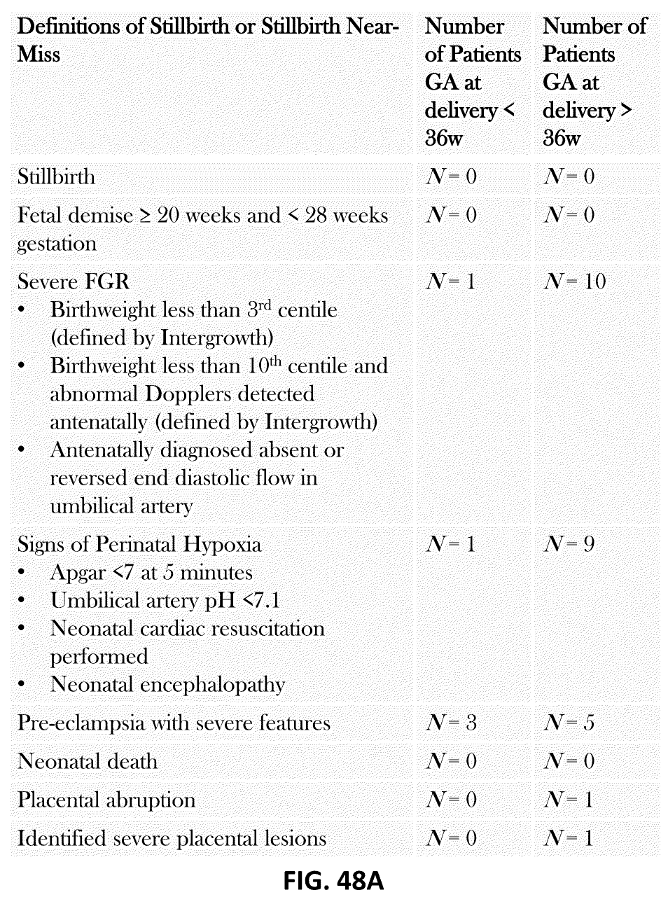

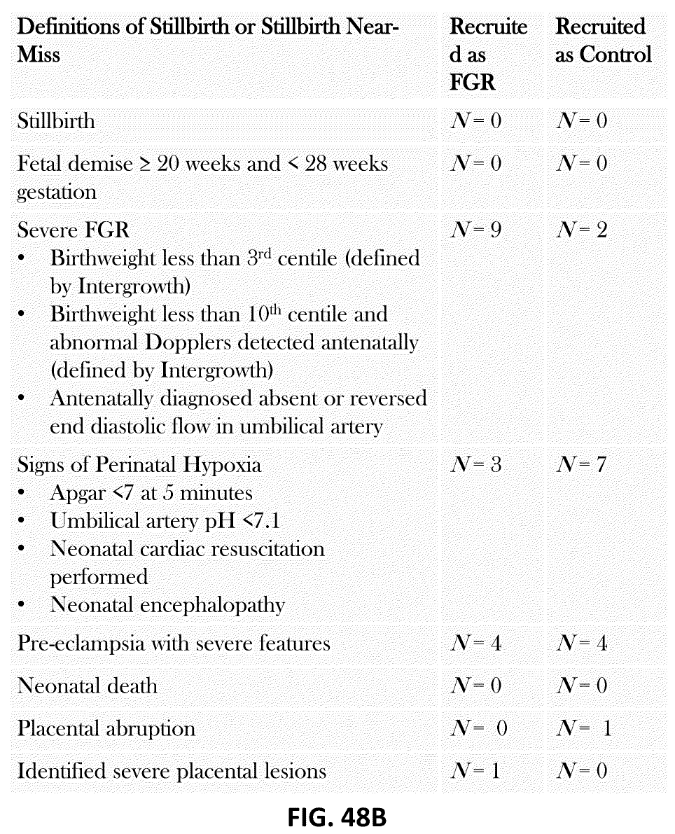

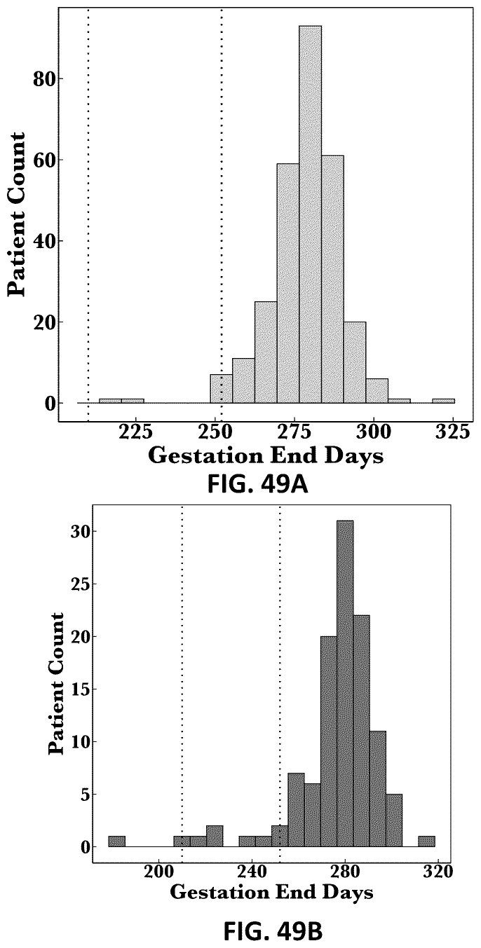

Additional texture image analysis was performed to compare the non-FGR group to the severe FGR, severe pre-eclampsia (PE) and stillborn near-miss groups. Differences in the empirical distribution functions (EDF) of the non-FGR and severe FGR groups (FIGS. 29A and 29B), the non-FGR and severe pre-eclampsia (PE) groups (FIGS. 29C and 29D), and the non-FGR stillborn near-miss groups (FIGS. 29E and 29F) were observed. Other example of texture image analysis results for the non-FGR and severe pre-eclampsia (PE) groups are illustrated in FIGS. 30A-30I; differences in the empirical distribution functions (EDF) were observed for all texture parameters shown. FIG. 30J is a table summarizing the texture features that were significantly different between the non-FGR and severe pre-eclampsia (PE) groups.

Microvascular structure quantification was also performed. This includes collecting placental biopsies, performing OCT imaging on the biopsy, image segmentation, 3D reconstruction, and 3D structure quantification (FIG. 31). Surface area per volume (FIG. 32A), villi thickness (FIG. 32B), and number of pores per volume (FIG. 32C) were quantified for the control, other, near miss, and severe FGR groups. Differences between the patient groups were observed in the surface area per volume and the villi thickness (FIG. 34A and 34B; N=129 Patients; 66 Control, 37 Other, 15 Stillbirth Near-miss, 11 Severe FGR; *p<0.05, **p<0.01). It was also found that OCT measurements correlated with birthweight (FIG. 33), independent ultrasound and OCT measurements were correlated (FIG. 35), and independent US texture and OCT measurements were correlated (FIG. 36).

The OCT imaging was similarly collected and analyzed for the control, other, pre-eclampsia (PE) and severe PE groups. Volume fraction (FIG. 32D), villi thickness (FIG. 32E) and number of pores per volume (FIG. 32F); no significant differences were observed between these groups.

Logistic regression of Doppler ultrasound data to predict severe FGR and stillbirth near-miss. ROC curves with an AUC of 0.97 (accuracy: 93.8 ±3%, precision: 71.3±2%, recall: 83.3±2%) were produced to predict severe FGR (FIG. 37A), and severe FGR features were identified (FIG. 37B). Similarly, ROC curves with an AUC of 0.76 (accuracy: 77.5±5%, precision: 44.5±13%, recall: 42±18%) were produced to predict stillbirth near-miss cases (FIG. 37C), and stillbirth near-miss features were identified (FIG. 37D). These data were produced with a patient cohort of 122-129 patients using Doppler ultrasound data, minimal clinical dataset (pregnancy data), and texture analysis features. Validation was performed using stratified k-fold method (k=5).

A classification model for ultrasound images was developed, wherein a 77.78% accuracy was observed using 141 training images and 36 testing images (FIG. 38A). A weighted binary cross-entropy loss equation (FIG. 38B) was used to compensate for unbalanced data and threshold optimization was performed. With a 0.1 threshold, 5 true positives, 7 false positives, 1 false negative, and 23 true negatives were identified. With a 0.5 threshold, 4 true positives, 6 false positives, 2 false negatives, and 24 true negatives were identified.

Then, digital biomarker vision and clinical implementation is proposed (FIG. 39A), where a balance between a clinical perspective, including the number of lives saved and the ease of implementation, and the available data, including the quantity of data and the training data available, is described (FIG. 39B). Potential timelines for clinical implementation are proposed (FIG. 40A) based on the stillbirth near-miss, not stillbirth near-miss, and overall stillbirth near-miss population cohorts (FIG. 40B-C) as well as the distribution of the absolute value of Pearson's correlation coefficient (texture features to birthweight) on visit 1 and 2 of pregnant women (FIG. 40D). The overall goal of the implementation is to optimize predictive models to detect severe FGR or stillbirth of patients around 36 weeks of gestation. Based on stillbirth near-miss features (FIG. 41A) and the described supporting data (FIG. 41B), predicting pathologies that lead to stillbirth that are associated with changes in the microstructure of the placenta is also proposed.

The predictive model can be optimized with various degrees of clinical accessibility. This can include questions answered by the mother, and requires a simple exam, medical tests prior to pregnancy, and a simple exam during pregnancy. With larger datasets of stillbirth near-miss features (FIG. 41A), stillbirth near-miss placenta structural phenotypes can be identified, and an informed design of a mechanistic predictive model can be produced (FIG. 41B).

Further analysis gave additional evidence that independent US Doppler and ultrasound texture measurements are correlated (FIG. 43), texture analysis of ultrasound images of the segmented placenta was also performed (FIG. 44). Additionally, boundary erosion sensitivity analysis on 60% of the placenta was performed using both correlation sensitivity analysis (FIG. 45A-D) and energy sensitivity analysis (FIG. 45E-H).

Further analysis of left uterine artery PI (FIG. 46A), intervillous space PI (FIG. 46B), and spiral artery PI (FIG. 46C) as a function of gestational age between distinct patient groups from Doppler ultrasound was performed. Further logistic regression of Doppler ultrasound data to predict severe FGR and stillbirth near-miss were performed. ROC curves with AUC of 0.75 (accuracy: 87.6±5%, precision: 35±37%, recall: 26.7±22%) to predict severe FGR were produced (FIG. 47A), and severe FGR features were identified (FIG. 47B). Similarly, ROC curves with AUC of 0.76 (accuracy: 78.2±5%, precision: 42.7±12%, recall: 42.6±15%) to predict stillbirth near-miss were produced (FIG. 47C), and still-birth near-miss features were identified (FIG. 47D). The cohort included 122-129 patients, and prediction was performed using Doppler ultrasound data (no EFW), minimal clinical dataset (pregnancy data), texture analysis features. Validation was performed using a stratified k-fold method (k=5).

A slightly different logistic regression of Doppler ultrasound data was used to predict severe FGR and stillbirth near-miss. ROC curves with AUC of 0.75 (accuracy: 85.2±8%, precision: 30.6±4%, recall: 30±2%) to predict severe FGR were produced (FIG. 47E), and severe FGR features were identified (FIG. 47F). Similarly, ROC curves with AUC of 0.67 (accuracy: 70.5±6%, precision: 31.1±12%, recall: 34±12%) to predict stillbirth near-miss were produced (FIG. 47G), and still-birth near-miss features were identified (FIG. 47H). The cohort included 122-129 patients, and prediction was performed using minimal clinical dataset (pregnancy data) and texture analysis features. Validation was performed using a stratified k-fold method (k=5).

Finally, still-birth near-miss patients were characterized in 2 tables (FIG. 48A and 48B), and clinical implementation of data of cohorts of controls (FIG. 49A), SFGR (FIG. 49B), and still-birth near-miss (FIG. 49C) in relation to gestation weeks is provided.

What is claimed is:

1. A computer-implemented system for predicting fetal development, maternal health, and any combination thereof, the system comprising at least one processor operatively coupled to a non-volatile memory, wherein the at least one processor is configured to:
a. receive an ultrasound image of a placenta of a subject;
b. transform the ultrasound image into at least one texture parameter of the subject; and
c. predict the fetal development, maternal health, and any combination thereof based on the at least one texture parameter of the subject.

2. The system of claim 1, wherein the at least one ultrasound image of a placenta comprises an ultrasound image segmented to isolate the placenta image.

3. The system of claim 1, wherein the at least one texture parameter of the subject is obtained using a texture analysis method selected from a filter-based method, a spectral method, a structural method, a deep learning method, and any combination thereof.

4. The system of claim 1, wherein the at least one texture parameter of the subject comprises at least one metric of a Gray Level Co-occurrence Matrix (GLCM) selected from contrast, dissimilarity, homogeneity, energy, correlation, and any combination thereof.

5. The method of claim 1, wherein predicting the fetal development, maternal health, and any combination thereof based on the at least one texture parameter further comprises predicting a fetal abnormality comprising a fetal growth restriction (FGR) or a maternal abnormality comprising a severe pre-eclampsia (PE) condition.

6. The method of claim 5, wherein the FGR condition or severe PE condition is predicted based on a comparison of at least one metric of a Gray Level Co-occurrence Matrix (GLCM) obtained from the ultrasound image of the subject, a healthy subject, a reference subject with a known FGR condition, and a reference subject with a known severe PE condition.

7. The method of claim 6, wherein:
a. an FGR condition is predicted when the at least one metric of the Gray Level Co-occurrence Matrix (GLCM) is significantly different from a corresponding metric of the healthy reference subject and the reference subject with the known PE condition; and
b. a severe PE condition is predicted when the at least one metric of a Gray Level Co-occurrence Matrix (GLCM) is significantly different from a corresponding metric of the healthy reference subject and the reference subject with the known FGR condition.

8. The method of claim 6, wherein FGR is predicted when:
a. the contrast of the ultrasound image is lower than a corresponding contrast of the reference healthy subject;
b. the dissimilarity of the ultrasound image is lower than a corresponding dissimilarity of the reference healthy subject;
c. the homogeneity of the ultrasound image is higher than a corresponding homogeneity of the reference healthy subject; and
d. the energy of the ultrasound image is higher than a corresponding energy of the reference healthy subject.

9. The method of claim 1, wherein the ultrasound image is a placental image of the subject.

10. A computer-implemented system for selecting a treatment for a pregnant subject based on an ultrasound image of a placenta of the subject, the system comprising at least one processor operatively coupled to a non-volatile memory, wherein the at least one processor is configured to:
a. receive the ultrasound image of the placenta of the subject;
b. transform the ultrasound image into at least one texture parameter;
c. predict a fetal abnormality comprising a fetal growth restriction (FGR) or a maternal abnormality comprising a severe pre-eclampsia (PE) condition Fetal Growth Restriction based on the at least one texture parameter; and
d. recommend the treatment if the FGR or PE condition is predicted, wherein the treatment comprises an FGR treatment or a PE treatment.

11. The system of claim 10, wherein the at least one ultrasound image of a placenta comprises an ultrasound image segmented to isolate the placenta image.

12. The system of claim 10, wherein the at least one texture parameter is obtained using a texture analysis method selected from a filter-based method, a spectral method, a structural method, a deep learning method, and any combination thereof.

13. The system of claim 10, wherein the at least one texture parameter comprises at least one metric of a Gray Level Co-occurrence Matrix (GLCM) selected from contrast, dissimilarity, homogeneity, energy, correlation, and any combination thereof.

14. The method of claim 13, wherein the FGR is predicted based on a comparison of at least one metric of a Gray Level Co-occurrence Matrix (GLCM) obtained from the ultrasound image and from a healthy subject.

15. The method of claim 14, wherein:
a. an FGR condition is predicted when the at least one metric of the Gray Level Co-occurrence Matrix (GLCM) is significantly different from a corresponding metric of the healthy reference subject and the reference subject with the known PE condition; and
b. a severe PE condition is predicted when the at least one metric of a Gray Level Co-occurrence Matrix (GLCM) is significantly different from a corresponding metric of the healthy reference subject and the reference subject with the known FGR condition.

16. The method of claim 14, wherein the FGR condition is predicted when:
a. the contrast of the ultrasound image is lower than a corresponding contrast of a healthy subject;
b. the dissimilarity of the ultrasound image is lower than a corresponding dissimilarity of a healthy subject;
c. the homogeneity of the ultrasound image is higher than a corresponding homogeneity of a healthy subject; and
d. the energy of the ultrasound image is higher than a corresponding energy of a healthy subject.

17. The method of claim 9, wherein:
a. the FGR treatment is selected from regular monitoring of fetal growth and well-being, recommending delivery before an expected due date, administering a corticosteroid compound to accelerate fetal lung development, maternal hospitalization for closer observation and management, recommending specialized neonatal care, and any combination thereof; and
b. the pre-eclampsia treatment is selected from regular monitoring of maternal blood pressure and fetal well-being, administering an eclampsia-preventing compound comprising magnesium sulphate, administering an antihypertensive compound to control maternal blood pressure, administering a corticosteroid compound to accelerate fetal lung development, and any combination thereof.

18. The method of claim 17, wherein the antihypertensive compound is selected from children's aspirin, labetalol, methyldopa, nifedipine, and any combination thereof.
